(19) United States
(12) Patent Application Publication
(10) Pub. No.: US 2007/0031876 A1
(43) Pub. Date: Feb. 8, 2007

(54) Methods of providing gene expression profiles for metastatic cancer phenotypes utilizing differentially expressed transcripts associated with circulating tumor cells

(76) Inventors: Hsueh-Kung Lin, Edmond, OK (US); Bradley P. Kropp, Edmond, OK (US)

Correspondence Address: DUNLAP, CODDING & ROGERS P.C., PO BOX 16370, OKLAHOMA CITY, OK, 73113, US

(21) Appl. No.: 11/500,631

(22) Filed: Aug. 8, 2006

Related U.S. Application Data
(60) Provisional application No. 60/706,696, filed on Aug. 8, 2005.

(51) Int. Cl. C12Q 1/68; C12N 1/08

(52) U.S. Cl. 435/6; 435/270

(57) ABSTRACT

Methods of identifying the presence of differentially expressed transcripts associated with the presence of at least one circulating tumor cell in a biological sample of a cancer patient involves the use of suppression subtractive hybridization to identify differentially expressed transcripts present in biological samples of cancer patients not present in healthy patients, as well as differentially expressed transcripts present in biological samples of healthy patients and not present in the cancer patients. Utilizing these methods, a gene expression profile associated with a specific cancer phenotype can be constructed.

### CROSS REFERENCE TO RELATED APPLICATIONS

This application claims benefit under 35 U.S.C. 119(e) of U.S. Ser. No. 60/706,696, filed Aug. 8, 2005, the contents of which are hereby expressly incorporated herein by reference in their entirety.

### STATEMENT REGARDING FEDERALLY SPONSORED RESEARCH AND DEVELOPMENT

Not applicable.

### BACKGROUND OF THE INVENTION

Advances in diagnostic techniques allow for cancer detection at earlier stages. However, metastasis, the spread of cancer from the primary tumor to other parts of the body, remains a problem in the search for a cure for cancer. Although metastasis of a solid tumor correlates well with the presence of tumor cells in the systemic circulation, genes that are responsible for the escape of tumor cells from primary organs, the survival of tumor cells in the circulation, and their development into metastatic disease are unknown. Current methods for detecting circulating tumor cells (CTCs) utilizes epithelial cell markers; however, these methods are complicated by the presence of a small number of circulating epithelial cells in healthy individuals as well as the potential loss of certain epithelial cell markers in CTCs. Therefore, it is essential that methods of identifying “tumor-specific genes” expressed by CTCs be discovered, thus allowing for early detection, disease management, and understanding of cancer metastasis. The present invention is directed to a genome-wide, comprehensive analysis of gene expression that offers an opportunity to catalogue genes whose expression is limited to CTCs, and thus monitor the progression of tumors.

Transformation of cells to an oncogenic phenotype initially occurs in defined organs and leads to dysregulated cell proliferation and organ-confined localized tumorigenesis. Subsequently, tumor cells detach from primary sites and metastasize into distant organs. Progression from tumorigeneis to metastasis is comprised of several intermediate steps, including the detachment of tumor cells into the local microvascular environments, survival of CTCs in the systemic circulation, and development of metastatic capability.

In order to metastasize, tumor cells must leave primary sites and invade the bloodstream, bone marrow, and/or lymph nodes. Tumor cells in bone marrow and lymph nodes are usually present at the time of primary tumor diagnosis, have a long survival time, and are usually dormant. In contrast, tumor cells in the peripheral blood are more dynamic. In order to survive in the bloodstream and spread, disseminated tumor cells' genomic DNA, mRNA, and protein expression levels must be altered with distinct cell populations. These alterations make the peripheral blood CTCs distinguishable from the majority of surrounding hematological cells. Since tumor cells are almost genetically heterogenous, searching for “common” genomic changes in tumor cells is difficult. There must be a common mechanism for CTCs to survive and develop metastatic capacity in the bloodstream.

Histological, immunological, and molecular techniques have been used to detect the presence of CTCs in the blood stream and to study the biology of these cells. However, to date, the number of well-characterized molecular tumor markers available for detecting occult cancer cells in blood are limited. Imaging and immunological techniques, such as but not limited to, tumor cell morphology, flow cytometry, cytogenetics, and immunocytochemistry have been used to detect peripheral blood CTCs. The use of these techniques for identification and detection of CTCs relies on antibody recognition of candidate “tissue-specific” or “tumor-specific” markers such as but not limited to, cytokeratins, CD45 (a tyrosine phosphatase), carcinoembryonic antigen (CEA), urokinase plasminogen activator receptor (uPA-R/CD87), prostate specific antigen (PSA), or prostatic acid phosphatase (PAP). However, these types of antibody-based techniques can yield false positive results if the expression of target molecules is shared by the host's benign epithelial cells, immune cells, or hematopoietic stem cells.

The highly sensitive reverse transcription polymerase chain reaction (RT-PCR) technique has greatly facilitated the sensitivity of detecting one tumor cell in 1-10×10⁶ normal cells in peripheral blood. This technique is based on the design of oligonucleotide primers that recognize the target genes of interest, and has provided a specific and sensitive tool to distinguish cells based on differential gene expression or genetic profiling.

Current RT-PCR based CTC detection is intended to detect two categories of molecules: general tumor cell characteristics (i.e., epithelial cell markers) and tissue-specific markers (i.e., PSA). Epithelial cell-specific molecules have been used to detect CTCs in at least 18 different solid tumors, including prostate cancer, breast cancer, malignant melanoma, gastrointestinal carcinoma, renal carcinoma, bladder cancer, testicular cancer, and lung cancer. In addition, PSA; prostate specific membrane antigen (PSMA); glandularkallikrein 2 (hK2); PTI-1; and HER-2/neu have been investigated for detecting prostate cancer cells in the circulation. Unfortunately, results are still inconclusive using these known markers.

All of the current techniques for detecting CTCs require enrichment of CTCs from peripheral blood. Enrichment is achieved through either density gradient or immunomagnetic depletion procedures relying mainly on epithelial cell markers. However, these protocols recover only a fraction of the potentially available CTCs, and the resulting cell populations are composed partially of non-tumor epithelial cells as well as dead or dying tumor cells. More importantly, cells separated from these techniques cannot be used for gene expression or biology studies since the lengthy separation procedures alter their gene expression patterns and cell physiology.

Tumor cell invasion into the bloodstream is an essential step for the systemic spread of cancer, and survival of tumor cells in the bloodstream is an essential step in cancer metastasis. It has been suggested that the majority of tumor cells entering the bloodstream are rapidly eliminated by factors such as but not limited to, blood turbulence, immune cells, or cytotoxic mediators such as nitric oxide secretion. In addition, the activation of apoptotic processes in disseminated tumor cells may contribute to the elimination of CTCs. Unfortunately, it is basically unknown how tumor cells escape from their primary sites, survive in the bloodstream, or gain metastatic capability.

The development of a genome-wide identification of tumor-specific genes is of utmost importance for all cancer development studies. Comprehensive gene expression analysis techniques have been attempted for studying and identifying CTCs. Twine et al. (2003) reported the use of oligo arrays for the comparison of separated peripheral blood mononuclear cells between healthy donors and patients with advanced renal cell carcinoma. Although sequences from the entire human genome can be spotted on a single microscope slide, microarray technology for sensitive detection of extremely diluted tumor cells in the circulation needs significant improvement.

In an attempt to identify “novel” tumor-specific markers as well as to understand the mechanisms of CTC survival and metastatic capability, Fournier et al. (1994) used mRNA differential display to screen genes that are expressed in separated peripheral blood mononuclear cells of patients with lung, breast, and colon cancer, but not expressed in healthy individuals. A total of 21 mRNA species have been found to be specifically expressed in mononuclear cells of cancer patients.

However, all current “tissue-specific” or “tumor-specific” markers neither specifically distinguish tumor cells from normal cells in peripheral blood, nor do these markers provide information regarding CTCs' survival or metastatic capability. In addition, all current methods require cell separation/enrichment steps that preferentially enrich certain populations of CTCs while excluding others, as well as introduce artifacts into the sample preparation steps and prevent the analysis of gene expression data. Current data have demonstrated that CTCs are present in the systemic circulation, and the presence of CTCs is positively correlated with cancer survival. Therefore, there is a need in the art for methods of identifying CTCs present in peripheral blood samples, thereby providing a “blood biopsy” screening method for the detection of cancer and/or metastasis.

### BRIEF DESCRIPTION OF THE DRAWINGS

FIG. 1 illustrates an evaluation of subtraction efficiency and the presence of potential differentially expressed genes in subtracted libraries constructed in accordance with the present invention. To determine subtraction efficiency, β-actin was PCR amplified using a primer set located within the very 3′-end of Rsal digested Mactin fragment following the second round of hybridization. PCR products were electrophoresed on an agarose gel. No Mactin product was detected after 40 cycles of PCR amplification in a subtracted library, whereas β-actin was detected after 25 cycles of amplification in a corresponding un-subtracted library (A). To amplify differentially expressed genes in circulating cells of healthy men and PCa patients, two rounds of PCR amplification were performed following the hybridization steps described in Methods. To demonstrate the presence of potential differentially expressed genes in the subtracted libraries, the final PCR products were analyzed on a 1.5% agarose gel followed by ethidium bromide staining. A series of distinct bands ranging from 300 to 1,000 bp were detected. These DNA fragments represented genes that are either present (B, lane 1) or absent (B, lane 2) in circulating cells of PCa patients.

FIG. 2 illustrates a virtual Northern blot analysis of genes that are specifically expressed in circulating cells of prostate cancer patients. Poly(A)+ RNA was isolated from whole blood using a mRNA isolation kit for blood/bone marrow. First-stranded cDNA synthesis and cDNA library amplification were performed in each individual patient sample using Clontech's switching mechanism at 5′ end of RNA transcript (SMART III™; Clontech Laboratories) protocol. An aliquot of 10 μl of the PCR products from each reaction was electrophoresed on a 1% agarose gel. The DNAs were denatured followed by neutralization and transferred onto the Hybond™N+nylon membranes. The membranes were hybridized with radiolabeled Pca-015 and cDNA fragments excised from the pCRII vector. The membrane was exposed to an x-ray film at −80° C. for autoradiography. The expression of PCa-015 is only present in circulating cells of prostate cancer patients but not those of healthy men.

FIG. 3 illustrates confirmation of differential gene expression in circulating cells of healthy men and PCa patients using semi-quantitative RT-PCR. RT-PCR was performed on individual samples from 8 healthy controls and 12 PCa patients to confirm the SSH results. After performing a sequencing reaction to reveal the identities of a total of 23 clones present in the subtracted libraries, PCR primers were designed (Table III). β-actin was also amplified from the same samples using a β-actin primer set (BD Biosciences Clontech) to serve as an internal control for standardizing the quantity of the RNA applied in each reaction. After PCR amplification, aliquots (10 μl) of these PCR products were electrophoresed on 2% agarose gels followed by ethidium bromide staining.

FIG. 4 illustrates relative levels of expression of target genes in peripheral blood circulating cells of healthy men and PCa patients. Images obtained from FIG. 3 were captured and analyzed using the Quantity One® software. For each target gene, levels of gene expression were normalized to the level of β-actin expression for each individual sample. * indicates statistical significance between healthy men and PCa patients at p<0.05.

### DETAILED DESCRIPTION OF THE INVENTION

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed.), Cold Spring Harbor Laboratory Press, Cold Spring Harbor, N.Y. (1989) and Ausubel et al. Current Protocols in Molecular Biology (Wiley Interscience (1988)), which are incorporated herein by reference. The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of animals.

As utilized in accordance with the present disclosure, the following terms, unless otherwise indicated, shall be understood to have the following meanings:

As used herein, the terms “nucleic acid segment” and “DNA segment” are used interchangeably and refer to a DNA molecule which has been isolated free of total genomic DNA of a particular species. Therefore, a “purified” DNA or nucleic acid segment as used herein, refers to a DNA segment which contains a coding sequence isolated away from, or purified free from, unrelated genomic DNA, genes and other coding segments. Included within the term “DNA segment”, are DNA segments and smaller fragments of such segments, and also recombinant vectors, including, for example but not by way of limitation, plasmids, cosmids, phage, viruses, and the like. In this respect, the term “gene” is used for simplicity to refer to a functional protein-, polypeptide- or peptide-encoding unit. As will be understood by those in the art, this functional term includes genomic sequences, cDNA sequences or combinations thereof. “Isolated substantially away from other coding sequences” means that the gene of interest forms the significant part of the coding region of the DNA segment, and that the DNA segment does not contain other non-relevant large portions of naturally-occurring coding DNA, such as large chromosomal fragments or other functional genes or DNA coding regions. Of course, this refers to the DNA segment as originally isolated, and does not exclude genes or coding regions later added to, or intentionally left in, the segment by the hand of man.

The term “a sequence essentially as set forth in SEQ ID NO:X” means that the sequence substantially corresponds to a portion of SEQ ID NO:X and has relatively few amino acids or codons encoding amino acids which are not identical to, or a biologically functional equivalent of, the amino acids or codons encoding amino acids of SEQ ID NO:X. For example, the sequence has less than 20 amino acids that are not identical to the amino acid sequence of SEQ ID NO:X, or the sequence has less than 15 amino acids that are not identical to the amino acid sequence of SEQ ID NO:X, or the sequence has less than 10 amino acids that are not identical to the amino acid sequence of SEQ ID NO:X, or the sequence has less than 5 amino acids that are not identical to the amino acid sequence of SEQ ID NO:X. The term “biologically functional equivalent” is well understood in the art and is further defined in detail herein, as a gene having a sequence essentially as set forth in SEQ ID NO:X, and that is associated with the ability to perform a desired biological activity in vitro or in vivo.

The art is replete with examples of practitioner's ability to make structural changes to a nucleic acid segment (i.e., encoding conserved or semi-conserved amino acid substitutions) and still preserve its enzymatic or functional activity when expressed. See for special example of literature attesting to such: (1) Risler et al. “Amino Acid Substitutions in Structurally Related Proteins. A Pattern Recognition Approach.” J. Mol. Biol. 204:1019-1029 (1988) [“ . . . according to the observed exchangeability of amino acid side chains, only four groups could be delineated; (i) Ile and Val; (ii) Leu and Met, (iii) Lys, Arg, and Gln, and (iv) Tyr and Phe.”]; (2) Niefind et al. “Amino Acid Similarity Coefficients for Protein Modeling and Sequence Alignment Derived from Main-Chain Folding Anoles.” J. Mol. Biol. 219:481-497 (1991) [similarity parameters allow amino acid substitutions to be designed]; and (3) Overington et al. “Environment-Specific Amino Acid Substitution Tables: Tertiary Templates and Prediction of Protein Folds,” Protein Science 1:216-226 (1992) [“Analysis of the pattern of observed substitutions as a function of local environment shows that there are distinct patterns . . . Compatible changes can be made.”]

These references and countless others indicate that one of ordinary skill in the art, given a nucleic acid sequence or an amino acid sequence, could make substitutions and changes to the nucleic acid sequence without changing its functionality. One of ordinary skill in the art, given the present specification, would be able to identify, isolate, create, and test DNA sequences and/or enzymes that produce natural or chimeric or hybrid molecules having a desired biological activity. As such, the presently claimed and disclosed invention should not be regarded as being solely limited to the specific sequences disclosed herein. Standardized and accepted functionally equivalent amino acid substitutions are presented in Table I.

TABLE I

| | Conservative and Semi- |
|---|---|
| Amino Acid Group | Conservative Substitutions |
| Nonpolar R Groups | Alanine, Valine, Leucine, Isoleucine, |
| | Proline, Methionine, |
| | Phenylalanine, Tryptophan |
| Polar, but uncharged, R Groups | Glycine, Serine, Threonine, Cysteine, |
| | Asparagine, Glutamine |
| Negatively Charged R Groups | Aspartic Acid, Glutamic Acid |
| Positively Charged R Groups | Lysine, Arginine, Histidine |

The terms “polypeptide,” “peptide,” and “protein”, used interchangeably herein, refer to a polymeric form of amino acids of any length, which can include coded and non-coded amino acids, chemically or biochemically modified or derivatized amino acids, and polypeptides having modified peptide backbones. The term includes polypeptide chains modified or derivatized in any manner, including, but not limited to, glycosylation, formylation, cyclization, acetylation, phosphorylation, and the like. The term includes naturally-occurring peptides, synthetic peptides, and peptides comprising one or more amino acid analogs. The term includes fusion proteins, including, but not limited to, fusion proteins with a heterologous amino acid sequence, fusions with heterologous and homologous leader sequences, with or without N-terminal methionine residues; immunologically tagged proteins; and the like.

As used herein, the term “anticancer agent” refers to a molecule capable of inhibiting cancer cell function. The agent may inhibit proliferation or may be cytotoxic to cells. A variety of anticancer agents can be used and include those that inhibit protein synthesis and those that inhibit expression of certain genes essential for cellular growth or survival. Anticancer agents include those that result in cell death and those that inhibit cell growth, proliferation and/or differentiation. In one embodiment, the anticancer agent is selectively toxic against certain types of cancer cells but does not affect or is less effective against other normal cells.

The term “antineoplastic agent” is used herein to refer to agents that have the functional property of inhibiting a development or progression of a neoplasm in a human or animal, particularly a malignant (cancerous) lesion, such as but not limited to, carcinoma, sarcoma, lymphoma, leukemia and the like. Inhibition of metastasis is frequently a property of antineoplastic agents.

The term “effective amount” refers to an amount of a biologically active molecule or conjugate or derivative thereof sufficient to exhibit a detectable therapeutic effect without undue adverse side effects (such as toxicity, irritation and allergic response) commensurate with a reasonable benefit/risk ratio when used in the manner of the invention. The therapeutic effect may include, for example but not by way of limitation, inhibiting the growth of undesired tissue or malignant cells. The effective amount for a subject will depend upon the type of subject, the subject's size and health, the nature and severity of the condition to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein. As anticancer agents and/or antineoplastic agents are widely known in the art, effective amounts thereof are also widely known and therefore use of such effective amounts of anticancer agents/antineoplastic agents falls within the scope of the present invention.

As used herein, the term “concurrent therapy” is used interchangeably with the terms “combination therapy” and “adjunct therapy”, and will be understood to mean that the patient in need of treatment is treated or given another drug for the disease in conjunction with the anticancer agents/antineoplastic agents in accordance with the present invention. This concurrent therapy can be sequential therapy where the patient is treated first with one drug and then the other, or the two drugs are given simultaneously.

The term “pharmaceutically acceptable” refers to compounds and compositions which are suitable for administration to humans and/or animals without undue adverse side effects such as toxicity, irritation and/or allergic response commensurate with a reasonable benefit/risk ratio.

By “biologically active” is meant the ability to modify the physiological system of an organism. A molecule can be biologically active through its own functionalities, or may be biologically active based on its ability to activate or inhibit molecules having their own biological activity.

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

The terms “cancer” and “cancerous” refer to or describe the physiological condition in mammals that is typically characterized by unregulated cell growth. Examples of cancer include but are not limited to, carcinoma, lymphoma, blastoma, sarcoma, leukemia and the like. More particular examples of such cancers include squamous cell cancer, small-cell lung cancer, non-small cell lung cancer, gastrointestinal cancer, pancreatic cancer, glioblastoma, cervical cancer, ovarian cancer, liver cancer, bladder cancer, hepatoma, breast cancer, colon cancer, colorectal cancer, endometrial carcinoma, salivary gland carcinoma, kidney cancer, renal cancer, prostate cancer, vulval cancer, thyroid cancer, hepatic carcinoma, various types of head and neck cancer, and the like.

The term “metastasis” as used herein will be understood to refer to the spread of cancer from a primary tumor to other parts of the body. Metastasis is a sequential, multistep process in which tumor cells detach from a primary tumor, migrate through the basement membrane and extracellular matrix, and invade the lymphatic and/or blood systems. This is followed by the establishment of secondary tumors at distant sites.

The term patient includes human and veterinary subjects. The term “mammal”, as used herein for purposes of treatment, refers to any animal classified as a mammal, including human, domestic and farm animals, nonhuman primates, and any other animal that has mammary tissue.

The term “healthy patient” or “healthy individual” are used herein interchangeably and will be understood to refer to a patient or individual who is otherwise healthy and free of cancer.

The term “suppression subtractive hybridization” or “SSH” refers to a genome-wide gene expression analysis technique used to selectively enrich differentially expressed mRNA species in two target populations, as disclosed and claimed in U.S. Pat. No. 5,759,822, issued to Chenchik et al. on Jun. 2, 1998; and U.S. Pat. No. 5,565,340, issued to Chenchik et al. on Oct. 15, 1996, the contents of each of which are hereby expressly incorporated herein by reference. SSH is a method based on suppressive PCR that allows for the creation of subtracted cDNA libraries for the identification of genes differentially expressed in response to an experimental stimulus (Gurskaya et al., 1996). The SSH technique ignores the abundance of mRNA species that are commonly expressed between samples under comparison and enriches target differentially expressed mRNA species through a sequential hybridization and suppression PCR. SSH differs from earlier subtractive methods by including a normalization step that equalizes for relative abundance of cDNAs within a target population. This modification enhances the probability of identifying increased expression of low abundance transcripts and represents a potential advantage over other methods for identifying differentially regulated genes, such as differential display-PCR (DD-PCR; Liang et al., 1992) and cDNA-representation difference analysis (Hubank et al., 1994). The methods of the present invention use the SSH procedure to successfully construct subtracted cDNA libraries comprising mRNA species that identify the presence of circulating tumor cells in the peripheral blood of patients with cancer.

The term “differential expression,” or grammatical equivalents as used herein, refers to both qualitative as well as quantitative differences in a genes' temporal and/or cellular expression patterns within and among cells. Thus, certain genes can qualitatively have their expression altered (including activation and inactivation) in, for example, normal versus cancerous cells or versus circulating tumor cells. That is, genes may be turned on or turned off in a particular state relative to another state. As is apparent to the skilled artisan, any comparison of two or more states can be made. Such a qualitatively regulated gene will exhibit an expression pattern within a state or cell type which is detectable by standard techniques in one such state or cell type, but is not detectable in both. Alternatively, the determination is quantitative in that expression is increased or decreased; that is, the expression of the gene is either upregulated, resulting in an increased amount of transcript, or downregulated, resulting in a decreased amount of transcript, when a gene's expression pattern within one state or cell type is compared to the gene's expression pattern in a different state or cell type. The degree to which expression differs need only be large enough to quantify via standard characterization techniques, such as but not limited to, quantitative reverse transcriptase PCR, Northern analysis, RNase protection, microarrays, and the like. In one embodiment, the change in expression (i.e., upregulation or downregulation) is at least about 30%, in another embodiment at least about 50%, in another embodiment at least about 100%, in yet another embodiment at least about 150%, in yet another further embodiment at least about 200%, and in yet another further embodiment from about 300 to at least about 1000%.

The term “cancer phenotype” as used herein will be understood to refer to a particular type of cancer, such as but not limited to, prostate cancer, breast cancer, bladder cancer, cervical cancer, ovarian cancer, squamous cell cancer, small-cell lung cancer, non-small cell lung cancer, gastrointestinal cancer, pancreatic cancer, glioblastoma, liver cancer, hepatoma, colon cancer, colorectal cancer, endometrial carcinoma, salivary gland carcinoma, kidney cancer, renal cancer, vulval cancer, thyroid cancer, hepatic carcinoma, and the like. In addition, the term “cancer phenotype” will also be understood to refer to a particular stage of a particular type of cancer, such as but not limited to, a contained prostate cancer, a potentially metastatic prostate cancer, a metastasized prostate cancer, a contained breast cancer, a potentially metastatic breast cancer, a metastasized breast cancer, etc.

The term “gene expression profile” will be understood to refer to an evaluation of the expression levels of genes compared between two different cellular or phenotypic states. As applicable to a certain cancer phenotype, a “gene expression profile”, as used herein, refers to a comparison of the expression levels of genes in a normal biological sample and in a biological sample from a cancer patient, and in some cases, to a comparison of two biological samples from cancer patients with varying severities of cancer that relate to prognosis, such as but not limited to, varying stages of metastasis. An expression profile of a particular cancer state or point of development is essentially a “fingerprint” of that state; while two states may have any particular gene similarly expressed, the evaluation of a number of genes simultaneously allows the generation of a gene expression profile that is unique to that cancerous state. By comparing expression profiles of samples obtained from cancer patients in different states versus samples from healthy patients, information regarding which genes are important (including both up- and down-regulation of genes) in each of these states is obtained. Then, diagnosis of a particular cancer, as well as diagnosis of the metastatic potential of that cancer, can be performed and/or confirmed, based on the gene expression profile of the patient's biological sample.

As will be appreciated by those in the art, gene expression profiles may be evaluated at either the gene transcript or the protein level; that is, the amount of gene expression may be monitored using nucleic acid probes to the DNA or RNA equivalent of the gene transcript, and the quantification of gene expression levels, or, alternatively, the final gene product itself (protein) can be monitored, for example through the use of antibodies to the differentially expressed protein, and standard immunoassays (ELISAs,etc.) or other techniques, including but not limited to, mass spectroscopy assays, 2D gel electrophoresis assays, and the like. Thus, the proteins corresponding to differentially expressed transcripts in a particular cancer phenotype can be evaluated in a diagnostic test. For example but not by way of limitation, a nucleic acid probe can be constructed to detect any of SEQ ID NOS:1-23 by methods well known in the art, orone or more antibodies can be raised against an amino acid sequence encoded by any of SEQ ID NOS:1-23 by methods well known in the art. The nucleic acid probes and/or antibodies could then be utilized in an assay to detect for one of the differentially expressed transcripts in a particular phenotype.

In one embodiment, gene expression monitoring is done and a number of genes, i.e., an expression profile, is monitored simultaneously, although multiple protein expression monitoring can be done as well. Similarly, these assays may be done on an individual basis as well.

The present invention provides novel methods for screening for compositions which modulate a cancer phenotype. As above, this can be done on an individual gene level or by evaluating the effect of drug candidates on a gene expression profile. In one embodiment, the gene expression profiles are used, such as in conjunction with high throughput screening techniques to allow monitoring for changes in gene expression profiles after treatment with a known anti-cancer agent or a candidate agent. Thus, the methods of the present invention allow for screening of candidate bioactive agents that may modulate expression of one or more genes in a gene expression profile of a cancer phenotype. The term “modulation” as used herein includes both an increase and a decrease in gene expression. The desired amount of modulation will depend on the original change of the gene expression in biological samples from normal versus cancer patients, with changes of at least 10%, in some cases at least 50%, in other cases at least 100-300%, and in some embodiments 300-1000% or greater desired.

As will be appreciated by those in the art, this may be done by evaluation at either the gene or the protein level; that is, the amount of gene expression may be monitored using nucleic acid probes and the quantification of gene expression levels, or, alternatively, the gene product itself can be monitored, for example through the use of antibodies to the gene expression products and standard immunoassays.

Thus, for example, a biological sample from a cancer patient in which circulating tumor cells have been identified may be screened for agents that reduce or suppress a cancer phenotype in which metastatic potential has been observed. A change in at least one gene of the expression profile indicates that the agent has an effect on the circulating tumor cells. By defining such a signature for the metastatic potential cancer phenotype, screens for new drugs that alter the phenotype can be devised.

Before explaining at least one embodiment of the invention in detail by way of exemplary drawings, experimentation, results, and laboratory procedures, it is to be understood that the invention is not limited in its application to the details of construction and the arrangement of the components set forth in the following description or illustrated in the drawings, experimentation and/or results. The invention is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

The present invention is directed to a method of identifying the presence of at least one differentially expressed transcript associated with the presence of at least one circulating tumor cell in a biological sample of a cancer patient. The method includes the steps of providing at least one biological sample from a patient diagnosed with a type of cancer and at least one biological sample from a healthy patient. The biological samples may be, for example but not by way of limitation, blood samples, such as peripheral blood samples, or bone marrow samples, lymph node samples, tissue samples and the like. The cells present in each of the biological samples are lysed without performing any cell separation or cell enrichment steps prior to lysing the cells, and total RNA or poly(A)+ RNA is then isolated from each of the lysed biological samples. Suppression subtraction hybridization is then performed to construct at least one subtracted cDNA library. It is determined that at least one circulating tumor cell is present in the at least one biological sample from a patient diagnosed with a type of cancer if: (1) at least one mRNA species is present in a substantially greater amount in the at least one biological sample from the patient diagnosed with a type of cancer when compared to the at least one biological sample from the healthy patient; or (2) at least one mRNA species is present in a substantially greater amount in the at least one biological sample from the healthy patient when compared to the at least one biological sample from the patient diagnosed with a type of cancer. The mRNA species detected in (1) above may comprise at least a portion of at least one of SEQ ID NOS:1-15, while the mRNA species detected in (2) above may comprise at least a portion of at least one of SEQ ID NOS:16-23.

The present invention is also directed to a method of providing a gene expression profile for a metastatic cancer phenotype, as well as the gene expression profile generated by such method. The method includes providing two or more biological samples from patients diagnosed with a type of cancer and combining such biological samples, and providing two or more biological samples from healthy patients and combining such biological samples. The cells present in the combined biological samples from patients diagnosed with a type of cancer are lysed without performing any cell separation or cell enrichment steps prior to lysing the cells, and the cells present in the combined biological samples from healthy patients are also lysed without performing any cell separation or cell enrichment steps prior to lysing the cells. Total RNA or poly(A)+ RNA is then isolated from the combined biological samples from patients diagnosed with a type of cancer and from the combined biological samples from healthy patients. Suppression subtraction hybridization is then performed to construct two subtracted cDNA libraries, wherein the first subtracted cDNA library comprises mRNA species present in a substantially greater amount in the combined biological samples from patients diagnosed with a type of cancer when compared to the combined biological samples from healthy patients, and wherein the second subtracted cDNA library comprises mRNA species present in a substantially greater amount in the combined biological samples from healthy patients when compared to the combined biological samples from patients diagnosed with a type of cancer. A gene expression profile is then constructed based on the differentially expressed mRNA species. The gene expression profile may comprise at least a portion of at least one of SEQ ID NOS:1-23. The gene expression profile may further include detection of at least a portion of at least one of SEQ ID NOS:1-15 in a substantially greater amount in the combined biological sample from patients diagnosed with a type of cancer when compared to the combined biological samples from healthy patients, and/or detection of at least a portion of at least one of SEQ ID NOS:16-23 in a substantially greater amount in the combined biological samples from healthy patients when compared to the combined biological samples from patients diagnosed with a type of cancer.

The present invention is also directed to a method of treating a patient by constructing a gene expression profile as described herein. The method may include providing at least one biological sample from the patient and providing at least one biological sample from a healthy individual. The cells in each of the biological samples are lysed without performing any cell separation or cell enrichment steps prior to lysing the cells, and total RNA or poly(A)+ RNA are then isolated from each of the biological samples. Suppression subtraction hybridization is then performed to construct at least one subtracted cDNA library, and it is determined that at least one circulating tumor cell is present in the at least one biological sample from the patient if at least one differentially expressed mRNA species selected from the following is present in the at least one subtracted cDNA library: (A) at least one mRNA species present in a substantially greater amount in the at least one biological sample from the patient when compared to the at least one biological sample from the healthy individual; and (B) at least one mRNA species present in a substantially greater amount in the at least one biological sample from the healthy individual when compared to the at least one biological sample from the patient. The method further includes treating the patient with an effective amount of an anticancer agent if at least one differentially expressed mRNA species is detected in the subtracted cDNA library. The at least one differentially expressed mRNA species detected may comprise at least a portion of at least one of SEQ ID NOS:1-23. The method may further include constructing a second subtracted cDNA library using the SSH technique described herein above following treatment with the anticancer agent to determine if the anticancer agent was effective in decreasing the number of circulating tumor cells.

The present invention is further directed to a method of identifying circulating tumor cells present in a biological sample from a patient. In one embodiment of the method, a biological sample is screened for expression of a mRNA species comprising at least a portion of at least one of SEQ ID NOS:1-23. In another embodiment of the method, a biological sample is screened for expression of a mRNA species comprising at least a portion of at least one of SEQ ID NOS:1-15, and/or screened for absence of expression of a mRNA species comprising at least a portion of at least one of SEQ ID NOS:16-23.

The present invention is further directed to kits that may be utilized in accordance with the present invention. In one embodiment, the kit includes an agent for detection of a mRNA species in a biological sample, wherein the mRNA species comprises at least a portion of at least one of SEQ ID NOS:1-15; the kit also includes a control sample that is substantially free of said mRNA species. The kit may further include a second agent for detection of a second mRNA species in a biological sample, wherein the mRNA species comprises at least a portion of at least one of SEQ ID NOS:16-23, and wherein the control sample is substantially free of said second mRNA species.

In another embodiment of the present invention, the kit includes an agent for detection of a first mRNA species comprising at least a portion of at least one of SEQ ID NOS:1-15, an agent for detection of a second mRNA species comprising at least a portion of at least one of SEQ ID NOS:16-23, and a control sample in which the second mRNA species can be detected and which is substantially free of the first mRNA species.

For any of the kits of the present invention, the agents for detection of a mRNA species may directly detect the mRNA species itself (such as but not limited to, a nucleic acid probe), or the agent may indirectly detect the mRNA species by directly detecting the expression product of the mRNA species (such as but not limited to, an antibody).

According to the present invention, methods of detecting the presence of at least one tumor cell in peripheral blood or other sample (such as but not limited to, bone marrow or lymph node), as well as methods of determining the stage of tumor development, are provided. The methods of the present invention include performing a comprehensive, genome-wide gene expression analysis of peripheral blood or other sample to identify differentially expressed transcripts. These differentially expressed transcripts may be present in circulating tumor cells (which may be present at very low concentrations in peripheral blood or other samples) from a sample of a cancer patient, and not present in a sample from a normal patient. In addition, the methods of the present invention may detect differentially expressed transcripts present in the sample from the normal patient but not in the sample from the cancer patient. These differentially expressed transcripts may be related to immune-specific gene changes that occur in response to the tumor.

The present invention is novel in that it does not require a cell separation step as used by methods of the prior art. This is important in that the circulating tumor cells may be present at very low concentrations in the peripheral blood or other sample from the cancer patient. For example, it is estimated that only one circulating tumor cell may be present for every 3 million circulating cells. Therefore, the present invention overcomes the disadvantages of the prior art, in which enrichment of the cell population results in loss of rare transcripts.

In addition, the current prior art methods for detecting circulating tumor cells rely on epithelial cell markers, based on the hypothesis that tumor cells continue to express genes of their origins. Therefore, the present invention overcomes this disadvantage of the prior art by detecting any transcripts expressed in circulating tumor cells of a cancer-bearing patient that are not expressed by circulating cells of a normal patient.

Another novel feature of the present invention is that the methods described herein detect not only the presence of tumor cells, but also detect alterations in gene expression patterns in immune cells in response to tumor development in cancer-bearing patients. The combination of detection of circulating tumor cells as well as detection of immune modulation in response to tumor development has important diagnostic, prognostic, and therapeutic implications.

Current protocols for detecting CTCs mainly utilize known epithelial cell markers or other tissue-specific molecules. However, the presence of these markers in CTCs does not correlate with the ability of CTCs to survive in the circulation or the metastatic capability of CTCs. Furthermore, molecules that participate in the process of “immunosurveillance” remain poorly understood. In the present invention, a PCR-based, genome-wide gene expression analysis named SSH was used to establish comprehensive, subtracted cDNA libraries to catalogue mRNA species either present or absent in circulating cells of PCa patients.

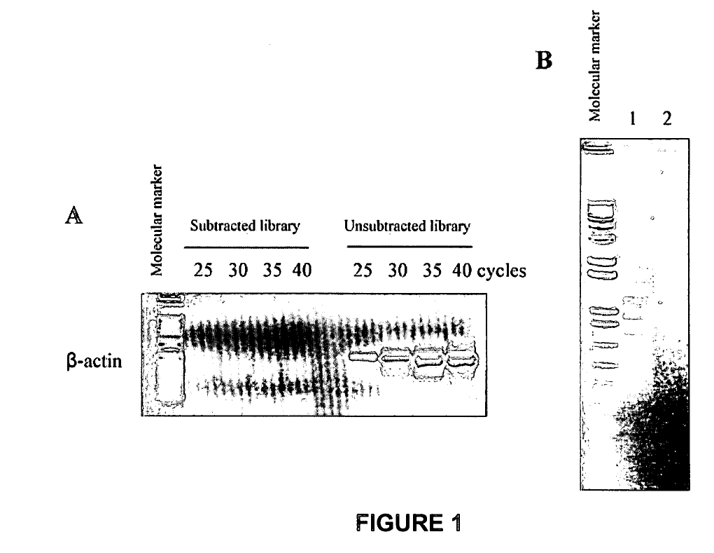

The SSH libraries were constructed from two age- and race-matched, pooled sample populations: (1) healthy men and (2) PCa patients. Each pooled sample included 25 individual double-stranded cDNA libraries derived from circulating cell poly(A)+ RNA of 25 men. A PCR-based method was used to evaluate hybridization efficiency. After two rounds of hybridization, β-actin was amplified from a subtracted population using a pair of gene-specific primers located within the very 3′ end of Rsal digested β-actin. No DNA product was detectable after 40 cycles of amplification (FIG. 1A), whereas the corresponding un-subtracted library showed the presence of abundant β-actin. This result demonstrated that β-actin, and therefore the commonly expressed genes between the two sample populations, formed heterohybrids, and could not be amplified using the suppression PCR technique of Diatchenko et al. (1996). To demonstrate that the subtracted cDNA libraries contain differently expressed mRNA species, the PCR products were electrophoresed on an agarose gel after the second round of PCR. A series of DNA fragments ranging from 300 to 1,000 bp in size and representing mRNA species specifically expressed in PCa patients, were detected. The various PCR products representing the Rsal digested cDNA fragments are shown in FIG. 1B, lane 1. These results indicated that there are differentially expressed genes present only in the circulating cells of PCa patients but not in the circulating cells of healthy men. PCR products representing genes expressed only in the circulating cells of healthy men are shown in FIG. 1B, lane 2.

To reveal the identities of the cDNA clones, the second round PCR products from both subtracted libraries were subcloned into the pCRII TA cloning vector. A total of 23 clones from both subtracted libraries were randomly selected for sequencing using M13 reverse primer. Identities of these clones are provided in SEQ ID NOS:1-23, and are listed in Table II. A majority of the sequenced clones, 17 out of 23 (73.9%), matched to portions of genomic DNA fragments in the GenBank database; however, none of these mRNA species had previously been identified. Therefore, the present invention is the first to indicate the presence of specific genes in these genomic DNA fragments, as well as indicate a potential function for these gene products in tumor progression. These results indicate that the methods of the present invention are capable of identifying rare or “novel” mRNA species that are present in an extremely small number of cells, thus reflecting that some of these clones were amplified from rare CTCs in PCa patients, and therefore that mRNA species reflecting these cells' biology or pathology have not been previously identified using traditional cDNA construction and sequencing. In addition, it is possible that genes identified from both subtracted libraries may represent previously un-identified molecules participating in tumor-immune system interactions (see for example, Nakano et al., 2001; Girardi et al., 2001; Kelly et al., 2002; and Diefenbach et al., 2003).

TABLE II
Identities of Selected cDNA Clones Present in Subtracted Libraries

| | SEQ ID | GenBank | |
|---|---|---|---|
| Clone I.D. | NO: | Accession No. | Gene Description for GenBank Accession No. |
| PCa-001* | 1 | AC026205 | Homo sapiens chromosome 3 clone RP11-61I9 map 3p, |
| | | | complete sequence |
| PCa-002 | 2 | AC019106 | Homo sapiens BAC clone RP11-479L11 from 2, |
| | | | complete sequence |
| PCa-004 | 3 | AY341247.1 | Homo sapiens integral membrane protein 2B (ITM2B) |
| | | | gene, complete cds |
| PCa-005 | 4 | AC 104771.4 | Homo sapiens BAC clone RP11-1E1 from 4, complete |
| | | | sequence |
| PCa-006 | 5 | AC132068 | Homo sapiens chromosome 16 clone CTD-2326c4, |
| | | | complete sequence |
| PCa-007 | 6 | AK091994 | Homo sapiens cDNA FLJ34675 fis, clone Liver2001608 |
| PCa-008 | 7 | AC092910.9 | Homo sapiens 3 BAC RP11-767L7 (Roswell Park Cancer |
| | | | Institute human BAC Library) |
| PCa-009 | 8 | AC 004690.2 | Homo sapiens PAC clone RP4-630C24 from 7, complete |
| | | | sequence |
| PCa-010 | 9 | AC097461 | Homo sapiens bCA clone RP11-16P6 from 2, complete |
| | | | sequence |
| PCa-011 | 10 | BC047553 | Homo sapiens calmodulin 2 mRNA (phosporylase kinase |
| | | | δ) |
| PCa-012 | 11 | AL031274 | Homo sapiens chromosome 1q24 (clone RP4-798A17) |
| | | | contains the 3′ end of the FMO1 gene and the FMO4 |
| | | | gene |
| PCa-013 | 12 | AC010369 | Homo sapiens chromosome 5 (clone CTD-2048F20) |
| PCa-014 | 13 | NG_002397 | Homo sapiens major histocompatibility complex, class I, |
| | | | BC (HLA-BC) |
| PCa-015 | 14 | BC016320 | Homo sapiens cathepsin D (Lysosomal aspartyl |
| | | | protease) |
| PCa-016 | 15 | AC021701 | Homo sapiens chromosome 18 (clone RP11-704G7) |
| Nrm1-001** | 16 | AC004914.1 | Homo sapiens PAC clone RP5-88608 from 7, complete |
| | | | sequence |
| Nrm1-002 | 17 | AK095899.1 | Homo sapiens cDNA FLJ38580 fis, clone |
| | | | HCHON20008582, highly similar to ferritin heavy chain |
| Nrm1-003 | 18 | AC 006083 | Homo sapiens chromosome 17, clone hRPK. 1053_B_8, |
| | | | complete sequence |
| Nrm1-004 | 19 | AL109759.4 | Human chromosome 14 DNA sequence BAC R-898B23 |
| | | | of library RPCI-11 from chromosome 14 of Homo |
| | | | sapiens (Human), complete sequence |
| Nrm1-005 | 20 | AK026823.1 | Homo sapiens cDNA: FLJ23170 fis, clone LNG09984 |
| Nrm1-006 | 21 | AC 019335 | Homo sapiens chromosome 8, clone RP11-453N18, |
| | | | complete sequence |
| Nrm1-007 | 22 | AL162252 | Human DNA sequence from clone RP11-55J24 on |
| | | | chromosome 9, complete sequence |
| Nrm1-008 | 23 | AC016644.9 | Homo sapiens chromosome 5 clone RP11-52M14, |
| | | | complete sequence |

*PCa clones were selected from the subtracted cDNA library that represents mRNA species only present in circulating cells of PCa patients.
**Nrml clones were selected from the subtracted cDNA library that represents mRNA species absent in circulating cells of PCa patients but present in their counterparts in cancer-free healthy men.

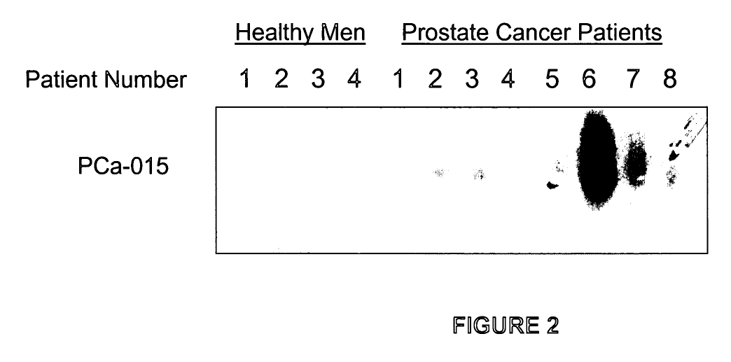

Since a portion of the subtracted cDNAs may be false positive clones (Diatchenko et al., 1996), it was necessary to confirm the identified clones as truly differentially expressed genes in the two sample populations. Two independent techniques were utilized to confirm clones in the SSH libraries specific for prostate cancer patients. The first technique used was virtual Northern blot analyses. Double-stranded cDNA libraries were generated from individual poly(A)⁺ samples (four healthy donors and eight prostate cancer patients) using a PCR-based SMART™ technology (Clontech). The resultant PCR products were electrophoresized on a 1% agarose gel, transferred onto Hybond+membrane (Amersham Biosciences), and immobilized with UV crosslinker (STRATALINKER®; Stratagene). The membrane was hybridized to [α-³²P]dATP-labeled, random primed, PCa-015 (Table II). Hybridization with the PCa-015 clone identified a single band in all prostate cancer samples that was not in any of the healthy donor samples (FIG. 2).

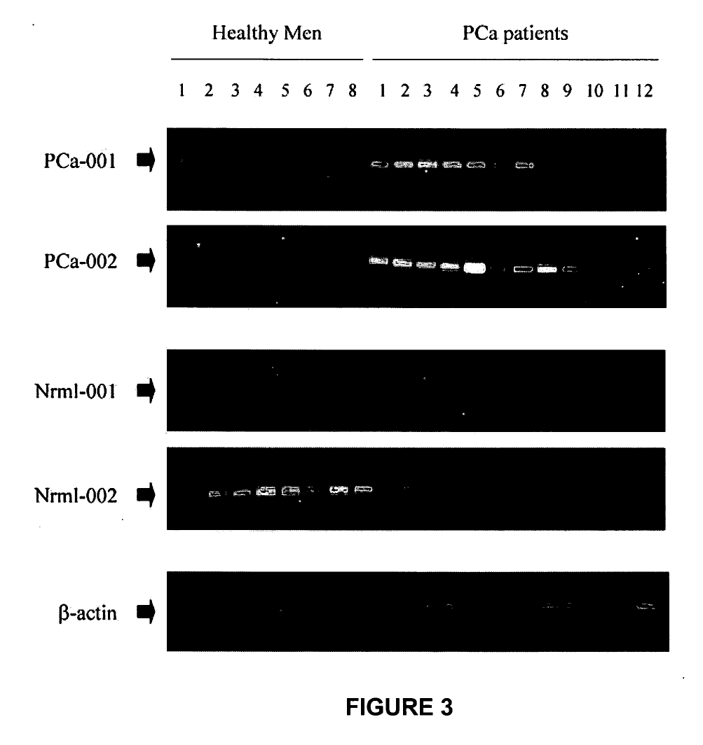

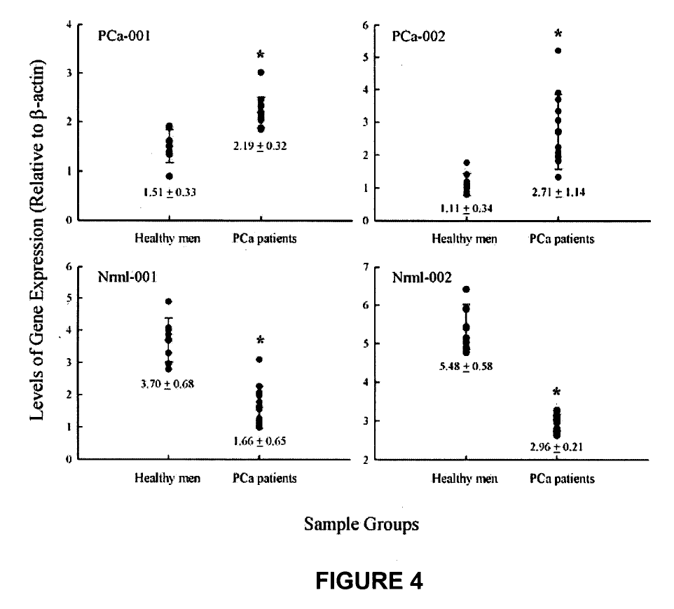

In addition, semi-quantitative RT-PCR was used to confirm that the cloned cDNAs were associated with peripheral blood circulating cells of cancer-bearing patients. Samples were collected from 12 PCa patients and 8 age- and race-matched healthy men for this analysis. RT-PCR was performed on individual samples. Total RNA was analyzed for selected genes for a total of 20 samples, 8 healthy men and 12 PCa patients. Four target genes were selected to be confirmed by RT-PCR. Two genes, PCa-001 and PCa-002, were selected from the library of mRNA species only present in circulating cells of PCa patients. PCR primers were designed according to the sequencing results (Table III), and the designed primers were subjected to a BLAST search to ensure that these sequences do not match any identified mRNA with high homology. As expected, PCa-001 and PCa-002 were expressed at significantly higher levels in circulating cells obtained from PCa patients than in healthy men (FIG. 3). The detection of low levels of PCa-001 and PCa-002 may be due to high sensitivity of the RT-PCR-based detection method. Martin et al. (2001) also reported low levels of Mdm-2 and Gro-alpha expression in peripheral blood mononuclear cells of healthy samples using RT-PCR, whereas array-based analysis did not show a detectible signal for these two genes. It is also possible that a very small number of epithelial cells can be present in the peripheral blood of healthy men (Garber, 2004), and PCa-001 and PCa-002 are epithelial cells markers. Another two genes, Nrml-001 and Nrml-002, were selected as being absent in PCa patients' circulating cells and present in circulating cells of healthy patients. These two genes were expressed, as expected, at higher levels in healthy men than in PCa patients (FIG. 3). Mactin has been shown to be constantly expressed in leukocytes of different individuals (Vandesompele et al., 2002). Therefore, it was demonstrated that the levels of β-actin expression were similar in all samples (FIG. 3). The relative levels of PCa-001, PCa-002, Nrml-001, and Nrml-002 expression were then normalized to β-actin, as illustrated in FIG. 4. Levels of these genes' expression were statistically different between healthy men and PCa patients.

TABLE III

PCR Primers and Conditions for Detecting Levels
of mRNA Expression in Circulating Cells of Healthy
Men and Patients with PCa

| | GenBank | | | SEQ |
|---|---|---|---|---|
| | Accession | | | ID |
| Clone I. D. | No. | PCR primers | cycles | NO: |
| PCa-0001 | AC132068 | 5′-AGG AAT AAG TCA CAC CGT GGA-3′ | 20 | 24 |
| | | 5′-ACC TGT TGG GAC TAG ACG CAT-3′ | | 25 |
| | nested | 5′-TGG TCT GTA ACC CTT AGG AGA-3′ | 25 | 26 |
| | | 5′-TCT GCC CTT TGA GTC CAA GT-3′ | | 27 |
| PCa-0002 | AC019106 | 5′-AGG TCA GCA GAG ATG TCT GT-3′ | 32 | 28 |
| | | 5′-TAG TCC CCG AGA AAG AAT TA-3′ | | 29 |
| Nrm-0001 | AC004690 | 5′-TGA GCA GTT TCT TCA GCC TCA-3′ | 20 | 30 |
| | | 5′-AGA GAC CAG CGT AAT ATC CCT-3′ | | 31 |
| | nested | 5′-TAT CTG GGT GAC ACT GGG AAA-3′ | 30 | 32 |
| | | 5′-AGA GAC CAG CGT AAT ATC CCT-3′ | | 33 |
| Nrm-002 | AK095899 | 5′-AGG TAA AGG AAA CCC CAA CAT GCA-3′ | 35 | 34 |
| | | 5′-AAC CAA CGA GGT GGC CGA ATC TT-3′ | | 35 |

In order to ensure that the PCR products were not the result of genomic DNA contamination, two pre-cautionary procedures were performed. First, all total RNA samples were subjected to RNase-free, DNase I digestion to remove residual genomic DNA. Second, a β-actin signal was detected after 26 cycles of PCR amplification in reactions with reverse transcription (FIG. 3), whereas a β-actin signal was absent in reactions without reverse transcription even after 35 cycles of amplification using amplimers within the same exon (data not shown).

The genome-wide screening and identification of mRNA species associated with circulating cells of tumor-bearing patients has been described for various cancers. Twine et al. (2003) reported the use of the microarray approach to differentiate gene expression patterns of mononuclear cells in patients with advanced renal cell carcinoma. However, due to the low number of tumor cells present in the circulation compared to the large number of normal leukocytes, hybridization array (cDNA array and oligonucleotide array) may not be an ideal tool for identifying rare molecular events occurring in a small number of CTCs. To overcome this problem, Smirnov et al. (2005) used magnetic separation of epithelial cell adhesion molecule (EpCAM) expressing cells from peripheral blood circulating cells and compared gene expression patterns between EpCAM-enriched and EpCAM-depleted cells using microarray analysis in cancer-bearing patients. Another comprehensive gene expression method, named mRNA differential display, has been conducted to compare genes that are expressed in peripheral blood mononuclear cells of tumor-free individuals with those separated blood cells from lung, breast, and colon cancer patients (Fournier et al., 1999). This study found a total of 21 mRNA species expressed in tumor patients' separated blood samples but not in samples from tumor free volunteers. In addition, Martin et al. (2001) reported the use of mRNA differential display to first identify transcripts differentially expressed in separated breast cancer cells and normal breast epithelial cells followed by an array analysis of these transcripts in separated circulating cells of breast cancer patients. However, all of these techniques required separation of the peripheral blood samples for detection of disseminated cancer cells.

It has been suggested that selective enrichment of the tumor cell population from both bone marrow and blood before analysis can increase the sensitivity for detecting CTCs (Racila et al., 2004; Gertler et al., 2003; Rosenberg et al., 2002; and Naume et al., 1998). Various cell separation techniques have been devised to enrich the CTC population from whole blood. However, these methods may also introduce artifacts into the sample preparation steps. For example, the addition of anticoagulants to blood samples affect leukocyte gene expression ex vivo (Riches et al., 1992; Freeman et al., 1990; and Rainen et al., 2002). In the methods of the present invention, efforts were made to avoid RNA degradation and alteration in gene expression during in vitro processing of blood cells and to avoid under- and over-estimation of in vivo mRNA expression. Direct isolation of poly(A)+ RNA and total RNA from whole blood was used to circumvent the need for a prior cell separation step. The methods of the present invention also prevent the loss of rare, un-identified target cells from blood samples during enrichment procedures since little is known about tumor cells' genotypes/phenotypes or any type(s) of immune cells' participation in cancer development.

Although it has been suggested that cancers are composed of a heterogeneous collection of cells with different degrees of expression of tumor markers (Baker et al., 2003; and Braun et al., 1999), CTCs of all types might need to develop a “common” mechanism(s) to survive in the circulation and acquire metastatic capability.

The tumor immunoediting theory states that the immune system responds to tumor development in three sequential steps: elimination, equilibrium, and escape. This theory emphases the tight relationship between immune cells and tumor development, progression, and evolution. Recent studies have repeatedly demonstrated that during tumor development and progression there are shifts in the composition of immune cell populations. These shifts include dendritic cells, macrophages, natural killer (NK) cells, and T cells locally and systematically in lymph nodes and peripheral blood. In addition, within each immune cell population, there are alterations in the expression of cell surface receptors and production of cytokines. However, the complex interactions between immune cells and tumors remain largely unknown. In the field of biomarker development for cancer detection, the primary focus has been on the identification of tumor cell-associated markers. A novel approach is the use of immune cell markers, as described herein.

The host immune system consists of innate and adaptive immune responses to protect the host from the attack of foreign entities, such as bacteria and viruses. The concept that the immune system can also recognize and eliminate developing tumors is called “cancer immunosurveillance”. Recently, this field has evolved into a concept that the immune system not only can protect the host against tumor development, it also has the capacity to promote tumor growth by selecting for tumors of lower immungenicity. The dual effects of the immune system on developing tumors were termed “tumor immunoediting”. Tumor cells can grow by “escaping” from the attack of immune cells through progressively “suppressing” the host immune system to allow tumor progression and metastasis. On the other hand, the immune system functions during tumor development, to select for tumor variants that are better suited to survive in an immunologically intact environment. However, the cause and effect of these tumor-immune cell interactions are unknown.

The understanding of tumor immunoediting is limited due to the lack of specific molecules involved in the process, although accumulated information has demonstrated the involvement of macrophages, NK cells, NKT cells, CD4+ T cells and CD8+ T cells in this process.

The cellular and molecular events by which cancer cells evade the host immune system have been investigated in in vitro models, animal models and human samples. Results from cancer immunology studies consistently suggest that immune cells are actively engaged with tumor cells locally and alterations of immune cell composition are present systemically.

A variety of tumor- and immune cell-derived factors have been attributed to the emergence of complex local tumor-immune cell networks. For example, human colorectal cancer cells express Fas as well as tumor necrosis factor (TNF)-related apoptosis-inducing ligands, and induce ligand-mediated apoptosis of activated CD8+ T cells (Huber et al., 2005). Tumor cells also cause dysfunction of cytotoxic T cells and NK cells through signaling proteins (Karimi et al., 2005; and Kiessling et al., 1999) and interleukin (IL)-2-mediated activation of NK cells, and their cytotoxic responses to tumor cells (Liu et al., 2006). In addition, dendritic cell maturation and regulatory T cell function are inhibited in response to the release of their own immunosuppressive cytokines, such as IL-10, transforming growth factor-β (TGF-β), and interferon-γ (IFN-γ), in tumor models and in human cancer patients (Liyanage et al., 2002; and Berger et al., 2005).

Tumor induced hematological alterations have been observed systemically in cancer bearing animals and humans. Tumor cells can recruit immature myeloid cells from bone marrow to become tumor-associated immature dendritic cells and immature macrophages through the peripheral blood circulation in a mouse model (Kimk et al., 2006; and Gabrilovich et al., 1998). The accumulation of the immature myeloid cell linages also appears in the peripheral blood (Almand et al., 2001). Immature myeloid cells are able to inhibit dendritic cell maturation and suppress T cell function (Song et al., 2005; Almand et al., 2000; and Kusmartsev et al., 2002). Several studies have reported that tumor-bearing patients possess shifts in T cell populations (Swann et al., 2004), such as increased frequencies of CD4⁺ CD25⁺ regulatory T cells (Somasundaram et al., 2002), in immune cell receptor expression (Girardi et al., 2003; and Smyth et al., 2005), and in immune cell cytokines production (Smyth et al., 2005; Tahir et al., 2001; and Russell et al., 2002).

Although some cellular events have been identified and proposed, both locally and systemically, during tumor development, molecules that regulate tumor and immune cell interactions are not yet understood. The tumor immunoediting network may result from elevated and/or suppressed gene expression in immune cells. For example, Jak2/STAT3 signaling and its downstream transcript factor NF-κB are responsible for the development of tumor-associated immature dendritic cells (Nefedova et al., 2005). Cataloguing alterations of gene expression profiling in peripheral blood cells is the first step toward an understanding of systemic alterations of immune cells in response to tumor development at the molecular level.

The present invention discloses that universal “tumor-specific” markers can be identified in occult tumor cells from different cancers. Moreover, “tissue-specific” molecules in CTCs can be identified if tumor cells continue to express their tissue-specific markers. The identification of tissue-specific markers will help to identify the origins of CTCs. The detection of tumor cells disseminated in peripheral blood can provide clinically important data for tumor staging, prognostication, and identification of surrogate markers for early assessment of the effectiveness of adjuvant therapy. Furthermore, by comparing gene expression profiling of all circulating cells, genes that might play a role in “immunosurveillance” can be identified. The methods of the present invention also allow for the identification of cell types that express differentially regulated mRNA species, and the study of the functional activities of these molecules in circulating cells during cancer development, thus establishing an association between these genes' expression and cancer stages.

In summary, the PCR-based SSH technique was used to establish two comprehensive subtracted cDNA libraries comprising differentially regulated genes in circulating cells of cancer-bearing patients. The methods of the present invention allow for the detection of both elevated and suppressed transcripts in circulating cells of cancer patients. The methods of the present invention allow a genome-wide gene expression analysis to be performed in peripheral blood circulating cells to demonstrate the presence of previously un-identified mRNA species in circulating cells of cancer-bearing patients. The methods of the present invention can be utilized in understanding tumor metastasis and tumor-induced immune reactions in the development of cancer, as well as to investigate these molecules' physiological and/or pathological function and their use in cancer detection.

Therefore, the methods of the present invention provide a genome-wide, comprehensive gene expression analysis to catalogue both “tumor-specific” markers as well as molecular changes in immune cells during tumor progression. Using the prostate cancer model presented herein, a person having ordinary skill in the art will easily be able to collect genes that are either suppressed or elevated in peripheral blood cells of cancer bearing patients as compared to those of healthy patients, and therefore the present invention is not limited to use with prostate cancer, but extends to all types of cancer for which a “blood biopsy” would be beneficial. Immune cell specific genes that directly or indirectly interact with the host's tumor cells and are responsible for the “tumor immunoediting” process during tumor progression can be identified by the methods of the present invention. It is believed that immune cells respond “identically” to all types of cancers, and thus the immune cell specific markers for tumor detection identified by the methods of the present invention have the advantage that these molecules are useful for multiple cancer types. Identification of alterations of immune cell-associated molecules in cancer development will strengthen and enhance not only cancer detection but also immunotherapy protocols for cancer prevention and treatment in the future.

### Methods

Patient selection: PCa patients were enrolled at the time of diagnosis of elevated PSA and positive biopsy. Healthy men's samples were collected from volunteers with similar age and race distribution without evidence of diseases or use of any medications. Attending physicians provided all participants with informed consent forms for collecting samples used in this study. Sample collection was also HIPAA compliant. Blood was drawn before scheduled surgery from PCa patients. There was no evidence of systemic metastases for all PCa patients when the primary tumor was resected through surgical prostatectomy. For initial construction of SSH libraries, we collected 50 samples, 25 healthy men and 25 patients with PCa. An additional 20 blood samples were collected from 8 healthy men and 12 patients with PCa, for RT-PCR analysis.

Blood collection and RNA isolation: For SSH, whole blood (5 ml) drawn from each individual was immediately mixed with 10× volume of RNA stabilization reagents for blood/bone marrow (Roche). The cells were then lysed. Poly(A)⁺ RNA was immediately isolated by a two-step procedure through magnetic separation using the mRNA isolation kit for blood/bone marrow (Roche). The poly(A)⁺ enriched samples were finally eluted from magnetic beads with H₂O. Purified poly(A)⁺ RNA was quantitated spectrophotometrically and stored in liquid nitrogen until use.

For RT-PCR, blood (2.5 ml) from each individual was colleted into a PAXgene™ Blood RNA tube (QIAGEN) following the manufacturer's protocol. Whole blood was thoroughly mixed with PAXgene stabilization reagent and stored at room temperature for 6 hours prior to RNA extraction. Total RNA was then extracted using the PAXgene™ Blood RNA kit according to the manufacturer's directions (QIAGEN). As the resulting RNA was usually contaminated with genomic DNA (Breit et al, 2004), total RNA samples absorbed to the PAXgene™ Blood RNA System spin column were incubated with DNase I (QIAGEN) at 25° C. for 20 min to remove genomic DNA. Total RNA was eluted, quantitated, and stored in liquid nitrogen.

Suppression subtractive hybridization (SSH) procedures: SSH was performed according to procedures described by Diatchenko et al. (1996). All reagents are now commercially available from BD Biosciences Clontech. Briefly, reverse transcription was performed with 2 μg poly(A)+ RNA from an individual patient sample in the presence of a mixture of three 3′ anchored primers (5′-TTTGCATGCTCGAG-(T)25-A/G/C-3′ (SEQ ID NO:36)) at 42° C. for 2 hours. Second strand cDNA was then synthesized with the addition of E. coli DNA polymerase 1 (250 μU/μl; Invitrogen), E. coli RNase H (8.5 μU/μl; Invitrogen), and E. coli DNA ligase (30 μU/μl; Invitrogen) at 16° C. for an additional 2 hours. The double-stranded cDNA libraries were then pooled into healthy and PCa groups. The pooled samples were subjected to Rsal digestion. To identify mRNA species expressed only in patients with PCa, the Rsal digested pooled cDNAs derived from PCa were ligated to specially designed adapters A and B (BD Biosciences Clontech) in two different reactions (Diatchenko et al., 1996).

To form heterohybrids between two sample populations, the adapter A and adaptor B ligated cDNAs (20 ng) were combined with excess Rsal digested cDNAs (400 ng) from healthy men in two separate reactions, heat-denatured, and hybridized at 68° C. for 10 hours. In a second hybridization step, the two separate samples from adapters A and B containing reactions were combined. A fresh aliquot of 150 ng heat-denatured Rsal digested cDNAs derived from healthy men was added to the combined reaction. Hybridization was continued for another 10 hours at 68° C. Commonly expressed sequences between controls and PCa patients formed hybrids in these two sequential hybridization steps. The heterohybrids are less likely to be amplified in the following PCR step due to the design of SSH adaptors (Diatchenko et al., 1996).

Genes specifically expressed in PCa patients' circulating cells were amplified by two consecutive rounds of PCR according to the procedures reported by Diatchenko et al., as disclosed in U.S. Pat. Nos. 5,759,822 and 5,565,340, previously incorporated herein by reference. The PCR-amplified products were then ligated to the pCRII vector (Invitrogen) followed by transformation. The bacteria were plated on agar plates containing ampicillin and overlaid with X-gal and IPTG. After overnight incubation, white colonies were picked and used for subsequent sequencing reaction. Sequencing results were used to design PCR primer sets to determine the genes' expression levels in healthy controls and PCa patients.

To detect sequences present in circulating cells of healthy men but absent in circulating cells of PCa patients, the initial adaptors ligation reaction was reversed. Aliquots of Rsal digested pooled cDNAs derived from healthy men were ligated to adapters A or B followed by hybridization and PCR amplification as described above.

RT-PCR confirmation: First strand cDNAs were reverse transcribed from 2.5 μg of the total RNA in the presence of oligo d(T) primer (Invitrogen), 20 μM each of dNTPs, and 200 units of M-MLV reverse transcriptase (Invitrogen). This was done in a total of 50 μl at 42° C. for 2 hours. PCR reactions were performed by mixing 1 μl of first-strand cDNAs, 0.2 μM gene-specific 5′ and 3′ primers (Table III), and 5 units Taq DNA polymerase (Invitrogen) in a total of 50 μl. Reactions were performed by heat activation at 94° C. for 2 min, followed by cycling through 94° C. for 30 sec, 50-55° C. for 1 min, and 72° C. for 1 min. The minimal numbers of PCR cycles required for detecting these gene products were first determined and is indicated in Table III. β-actin (NM_{—}001101) was also amplified and used as an internal control for comparing relative levels of target gene expression. RNA samples were also included without reverse transcription for β-actin amplification to determine levels of genomic DNA contamination. Following gel electrophoresis, images were captured using a Bio-Rad Gel Doc system; and band intensities were analyzed by the Quantity One® software (Bio-Rad).

Statistical Analysis: Levels of target gene expression were expressed as mean±standard deviation (SD) following normalization to β-actin. A Student's t test was used to compare means of these genes expressions between the healthy controls and PCa patients. A probability value of p<0.05 was considered significant.

Thus, in accordance with the present invention, there has been provided methods of screening and identification of differentially expressed transcripts associated with circulating tumor cells and immune modulation in response to cancer development that fully satisfies the objectives and advantages set forth hereinabove. Although the invention has been described in conjunction with the specific drawings, experimentation, results and language set forth hereinabove, it is evident that many alternatives, modifications, and variations will be apparent to those skilled in the art. Accordingly, it is intended to embrace all such alternatives, modifications and variations that fall within the spirit and broad scope of the invention.

### REFERENCES

The following references, to the extent that they provide exemplary procedural or other details supplementary to those set forth herein, are specifically incorporated herein by reference.
1. Ady N, Morat L, Fizazi K, Soria J C, Mathieu M C, Prapotnich D, Sabatier L, Chauveinc L. (2004) Br J Cancer, 90:443-448.
2. Baker M, Gillanders W E, Mikhitarian K, Mitas M, Cole D J (2003) Am J Surg, 186:351-358.
3. Blumke K, Bilkenroth U, Schmidt U, MelchiorA, Fussel S, Bartel F, Heynemann H, Fomara P, Taubert H, Wirth M P, Meye A (2005) Oncol Rep, 14:895-899.
4. Braun S, Hepp F, Sommer H L, Pantel K. (1999) Int J Cancer, 84:1-5.
5. Braun S, Pantel K (1999) Cancer Metastasis Rev, 18:75-90.
6. Breit S, Nees M, Schaefer U, Pfoersich M, Hagemeier C, Muckenthaler M, Kulozik A E. (2004) Br J Haematol., 126:231-243.
7. Burchill S A, Bradbury M F, Pittman K, Southgate J, Smith B, Selby P (1995) Br J Cancer 71:278-281.
8. Carrillo E, Marchal J A, Prados J, Melguizo C, Velez C, Arena N, Alvarez L, Serrano S, Aranega A (1998) Cell Mol Biol, 44:1247-1252.
9. Castells A, Boix L, Bessa X, Gargallo L, Pique J M (1998) Br J Cancer, 78:1368-1372.
10. Chambers A F, Naumov G N, Vantyghem S A, Tuck A B (2000) Breast Cancer Res, 2:400-407.
11. Cheung I Y, Sahota A, Cheung N K (2004) Cancer, 101:2303-2308.
12. Corey E, Arfman E W, Oswin M M, Melchior S W, Tindall D J, Young C Y, Ellis W J, Vesselia R L (1997) Urol, 50:184-188.
13. Courtemanche D J, Worth A J, Coupland R W, MacFarlane J K (1991) Can J Surg, 34:15-19.
14. Crnic I, Strittmatter K, Cavallaro U, Kopfstein L, Jussila L, Alitalo K, Christofori G (2004) Cancer Res, 64:8630-8638.
15. Dearnaley D P, Sloane J P, Ormerod M G, Steele K, Coombes R C, Clink H M, Powles T J, Ford H T, Gazet J C, Neville A M. (1981) Br J Cancer, 44:85-90.
16. Diatchenko L, Lau Y-F, Campbell A P, Chenchik A, Moqadam E, Huang B, Lukyanov S, Lukyanov K, Gurskaya N, Severdlov E D, Siebert P (1996) Proc Natl Acad Sci USA, 93:6025-6030.
17. Diefenbach A, Hsia J K, Hsiung M Y, Raulet D H. (2003) Eur J. Immunol., 33:381-391.
18. Dong Z, Staroselsky A H, Qi X, Xie K, Fidler, I J (1994) Cancer Res 54:789-793.
19. Dunn G P, Bruce A T, Ikeda H, Old L J, Schreiber R D. (2002) Nat Immunol., 3:991-998.
20. Dvorak H F (2002) J Clin Oncol, 20:4368-4380.
21. Eisenberger C F, Schoenberg M, Enger C, Hortopan S, Shah S, Chow N H, Marshall F F, Sidransky D (1999) J Natl Cancer Inst, 91:2028-2032.
22. Fabjani G, Tong D, Wolf A, Roka S, Leodolter S, Hoecker P, Fischer M B, Jakesz R, Zeillinger R (2005) Oncol Rep, 14:737-741.
23. Felton T, Harris G C, Pinder S E, Snead D R, Carter G I, Bell J A, Haines A, Kollias J, Robertson J F, Elston C W, Ellis I O. (2004) Breast, 13:3541.
24. Fidler I J (1985) Cancer Res, 45:4714-4726.
25. Fidler I J, Yano S, Zhang R D, Fujimaki T, Bucana C D (2002) Lancet Oncol, 3:53-57.
26. Fournier M V, Carvalho M G, Pardee A B (1999) Mol Med, 5:313-319.
27. Fournier M V, Guimaraes da Costa F, Paschoal M E, Ronco L V, Carvalho M G, Pardee A B (1999) Cancer Res, 59:3748-3753.
28. Franz O, Bruchhaus I, Roeder T (1999) Nucleic Acids Res, 27:e3.
29. Freeman R, Wheeler J, Robertson H, Paes M L, Laidler J (1990) Lancet, 336:312-313.
30. Fujii Y, Kageyama Y, Kawakami S, Kihara K, Oshima H. (1999) Jpn J Cancer Res., 90:753-757.
31. Garber K (2004) J Natl Cancer Inst., 96:1055-1057.
32. Gebauer G, Fehm T, Merkle E, Beck E P, Lang N, Jager W. (2001) J Clin Oncol., 19:3669-3674.
33. Gerhard M, Juhl H, Kalthoff H, Schreiber H W, Wagener C, Neumaier M. (1994); J Clin Oncol. 12:725-729.
34. Gertler R, Rosenberg R, Fuehrer K, Dahm M, Nekarda H, Siewert J R (2003) Cancer Res 162:149-155.
35. Ghossein R A, Bhattacharya S, Rosai J (1999) Clin Cancer Res 5:1950-1960.
36. Ghossein R A, Scher H I, Gerald W L, Kelly W K, Curley T, Amsterdam A, Zhang Z F, Rosai (1995), J. J Clin Oncol.;13:1195-1200.
37. Gilbey A M, Burnett D, Coleman R E, Holen I. (2004), J Clin Pathol. 57:903-911.
38. Girardi M, Oppenheim D E, Steele C R, Lewis J M, Glusac E, Filler R, Hobby P, Sutton B, Tigelaar R E, Hayday A C. (2001) Science., 294:605-609.
39. Glaves D (1983), Br J Cancer 48:665-673.
40. Gurskaya, N G, Diatchenko, L, Chenchik, A, Siebert, P D, Khaspekov, G L, Lukyanov, K A, Vagner, L L, Ermolaeva, O D, Lukyanov, S A, and Sverdlov, E D. (1996) Anal Biochem, 240:90-97.
41. Hanna N, Fidler I J (1980) J Natl Cancer Inst., 65:801-809.
42. Hildebrandt M, Mapara M Y, Komer I J, Bargou R C, Moldenhauer G, Dorken B. (1997) Exp Hematol.:25:57-65.
43. Hoon D S, Sarantou T, Doi F, Chi D D, Kuo C, Conrad A J, Schmid P, Turner R, Guiliano A. (1996) Int J Cancer., 69:369-374.
44. Hosch S B, Braun S, Pantel K (2001), Semin Surg Oncol 20:265-271.
45. Hsu C P, Shai S E, Hsia J Y, Chen C Y (2004) Cancer 100:794-800.
46. Hu X C, Wang Y, Shi D R, Loo T Y, Chow L W (2003), Oncology 64:160-165.
47. Israeli R S, Miller W H, Jr., Su S L, Samadi D S, Powell C T, Heston W D, Wise G J, Fair W R (1995) Cancer Res 153:573-577.
48. Johnson P W, Burchill S A, Selby P J (1995) Br J Cancer 72:268-276.
49. Jotsuka T, Okumura Y, Nakano S, Nitta H, Sato T, Miyachi M, Suzumura K, Yamashita J. (2004) Surgery. 135:419-426.
50. Jung R, Soondrum K, Neumaier M (2000) Quantitative PCR. Clin Chem Lab Med 38:833-836.
51. Kelly J M, Darcy P K, Markby J L, Godfrey D I, Takeda K, Yagita H, Smyth M J. (2002) Nat Immunol.;3:83-90.
52. Kirk S J, Cooper G G, Hoper M, Watt P C, Roy A D, Odling-Smee W. (1990) Eur J Surg Oncol 16:481-485.
53. Kopfstein L, Christofori G (2006), Cell Mol Life Sci 63:449-468.
54. Li X, Wong C, Mysel R, Slobodov G, Metwalli A, Kruska J, Manatt C S, Culkin D J, Kropp B P, Lin H K (2005) Mol Cancer 4:30.
55. Liang P, Averboukh L, Pardee A B (1993) Nucleic Acids Res 21:3269-3275.
56. Liang P, Pardee A B (1992) Science 257:967-971.
57. Lugo T G, Braun S, Cote R J, Pantel K, Rusch V (2003) J Clin Oncol 21:2609-2615.
58. Martin K J, Graner E, Li Y, Price L M, Kritzman B M, Foumier M V, Rhei E, Pardee A B (2001) Proc Natl Acad Sci USA 98:2646-2651.
59. Matsumura M, Niwa Y, Kato N, Komatsu Y, Shiina S, Kawabe T, Kawase T, Toyoshima H, Ihori M, Shiratori Y. (1994) Hepatology.; 20:1418-1425.
60. McKieman J M, Buttyan R, Bander N H, de la Taille A, Stifelman M D, Emanuel E R, Bagiella E, Rubin M A, Katz A E, Olsson C A, Sawczuk I S (1999), Cancer 86:492-497.
61. Meye A, Bilkenroth U, Schmidt U, Fussel S, Robel K, Melchior A M, Blumke K, Pinkert D, Bartel F, Linne C, Taubert H, Wirth M P (2002) Int J Oncol 21:521-530.
62. Miller W H Jr, Levine K, DeDeBlasio A, Frankel S R, Dmitrovsky E, Warrell RPJr (1993) Blood 82:1689-1694.
63. Moreno J G, Miller M C, Gross S, Allard W J, Gomella L G, Terstappen L W (2005) Urology 65:713-718.
64. Muller V, Stahmann N, Riethdorf S, Rau T, Zabel T, Goetz A, Janicke F, Pantel K (2005) Clin Cancer Res 11:3678-3685.
65. Nakano O, Sato M, Naito Y, Suzuki K, Orikasa S, Aizawa M, Suzuki Y, Shintaku I, Nagura H, Ohtani H. (2001) Cancer Res.; 61:5132-5136.
66. Naume B, Borgen E, Nesland J M, Beiske K, Gilen E, Renolen A, Ravnas G, Qvist H, Karesen R, Kvalheim G. (1998) Int J Cancer.; 78:556-560.
67. Noguchi S, Aihara T, Motomura K, Inaji H, Imaoka S, Koyama H. (1996) Am J Pathol.; 148:649-656.
68. Normanno N, De Luca A, Castaldo A, Casamassimi A, Di Popolo A, Zarrilli R, Porcellini A, Acquaviva A M, Awedimento V E, Pignata S (1998) Int J Oncol 13:443-447.
69. Nunlist E H, Dozmorov I, Tang Y, Cowan R, Centola M, Lin H-K (2004) J Steroid Biochem Mol Biol 91:157-170.
70. O'Hara S M, Moreno J G, Zweitzig D R, Gross S, Gomella L G, Terstappen L W (2004) Clin Chem 50:826-835.
71. Pantel K, Cote R J, Fodstad. (1999) J Natl Cancer Inst.; 91:1113-1124.
72. Pantel K, Riethmuller G. (1996) Curr Top Microbiol Immunol.; 213:1-18.
73. Pelkey T J, Frierson H F, Jr., Bruns D E (1996) Clin Chem 42:1369-1381.
74. Racila E, Euhus D, Weiss A J, Rao C, McConnell J, Terstappen L W, Uhr J W. (2004) Proc Natl Acad Sci USA.; 95:4589-4594.
75. Rainen L, Oelmueller U, Jurgensen S, Wyrich R, Ballas C, Schram J, Herdman C, Bankaitis-Davis D, Nicholls N, Trollinger D, Tryon V. (2002) Clin Chem.; 48:1883-1890.
76. Raj G V, Moreno J G, Gomella L G (1998) Cancer 82:1419-1442.
77. Redding W H, Coombes R C, Monaghan P, Clink H M, Imrie S F, Dearnaley D P, Ormerod M G, Sloane J P, Gazet J C, Powles T J, Munro Neville A (1983) Lancet, 2:1271-1274.
78. Retz M, Lehmann J, Roder C, Weichert-Jacobsen K, Loch T, Romahn E, Luhl C, Kalthoff H, Stockle M (2001) Eur Urol 39:507-515.
79. Riches P, Gooding R, Millar B C, Rowbottom A W (1992) J Immunol Methods 153:125-131.
80. Rosenberg R, Gertler R, Friederichs J, Fuehrer K, Dahm M, Phelps R, Thorban S, Nekarda H, Siewert J R (2002) Cytometry 49:150-158.
81. Salvadori B, Squicciarini P, Rovini D, Orefice S, Andreola S, Rilke F, Barletta L, Menard S, Colnaghi M I (1990) Eur J Cancer 26:865-867.
82. Sato T, Harao M, Nakano S, Jotsuka T, Suda N, Yamashita J (2005) Surgery 137:552-558.
83. Schmidt B, Anastasiadis A G, Seifert H H, Franke K H, Oya M, Ackermann R (2003) Anticancer Res 23:3991-3999.
84. Seiden M V, Kantoff P W, Krithivas K, Propert K, Bryant M, Haltom E, Gaynes L, Kaplan I, Bubley G, DeWolf W, Sklar J. (1994) J Clin Oncol.;12:2634-2639.
85. Shariat S F, Kattan M W, Song W, Bernard D, Gottenger E, Wheeler T M, Slawin K M. (2003) Cancer Res.; 63:5874-5878.
86. Siebert P D, Chenchik A, Kellogg D E, Lukyanov K A, Lukyanov S A (1995) Nucleic Acid Res. 23:1087-1088.
87. Singletary S E, Larry L, Tucker S L, Spitzer G (1991) J Surg Oncol 47:32-36.
88. Smimov D A, Zweitzig D R, Foulk B W, Miller M C, Doyle G V, Pienta K J, Meropol N J, Weiner L M, Cohen S J, Moreno J G, Connelly M C, Terstappen L W, O'Hara S M (2005) Cancer Res. 65:4993-4997.
89. Soeth E, Vogel I, Roder C, Juhl H, Marxsen J, Kruger U, Henne-Bruns D, Kremer B, Kalthoff H. (1997) Cancer Res.; 57:3106-3110.
90. Stahel R A, Mabry M, Skarin A T, Speak J, Bemal S D (1985) J Clin Oncol 3:455-461.
91. Stevens G L, ScheerWD, Levine E A (1996) Cancer Epidermiol Biomarkers Prev 5:293-296.
92. Stuehr D J, Nathan C F (1989) J Exp Med 169:1543-1555.
93. Taback B, Chan A D, Kuo C T, Bostick P J, Wang H J, Giuliano A E, Hoon D S (2001) Cancer Res 61:8845-8850.
94. Twine N C, Stover J A, Marshall B, Dukart G, Hidalgo M, Stadler W, Logan T, Dutcher J, Hudes G, Domer A J, Slonim D K, Trepicchio W L, Burczynski M E (2003) Cancer Res 63:6069-6075.
95. van Dongen G A, Brakenhoff R M, ten Brink C T, van Gog F B, de Bree R, Quak J J, Snow G B (1996) Anticancer Res 16:2409-2413.
96. Vandesompele J, De Preter K, Pattyn F, Poppe B, Van Roy N, De Paepe A, Speleman F (2002) Genome Biol 3:RESEARCH0034.
97. Wang Z P, Eisenberger M A, Carducci M A, Partin A W, Scher H I, Ts'o P O. (2000) Cancer; 88:2787-2795.
98. Weiss L (1992) Cancer Metastasis Rev 11:227-235.
99. Xie K, Huang S, Dong Z, Juang S H, Gutman M, Xie Q W, Nathan C, Fidler I J (1995) J Exp Med 181:1333-1243.
100. Yamamoto O, Takahashi H, Hirasawa M, Chiba H, Shiratori M, Kuroki Y, Abe S (2005) Respir Med 99:1164-1174.
101. Ylikoski A, Pettersson K, Nurmi J, Irjala K, Karp M, Lilja H, Lovgren T, Nurmi M (2002) Clin Chem 48:1265-1271.
102. Yuasa T, Yoshiki T, Tanaka T, Isono T, Okada Y (1999) Cancer Lett 143:57-62.
103. Zimmerman R A, Dozmorov I, Nunlist E H, Tang Y, Li X, Cowan R, Centola M, Frank M B, Culkin D J, Lin H-K (2004) Prostate Cancer Prostatic Dis 7:364-374.

What is claimed is:

1. A method of identifying the presence of at least one differentially expressed transcript associated with the presence of at least one circulating tumor cell in a biological sample of a cancer patient, the method comprising the steps of:
providing at least one biological sample from a patient diagnosed with a type of cancer;
providing at least one biological sample from a healthy patient;
lysing the cells present in each of the biological samples without performing any cell separation or cell enrichment steps prior to lysing the cells;
isolating total RNA or poly(A)+ RNA from each of the biological samples;
performing suppression subtraction hybridization to construct a subtracted cDNA library; and
determining that at least one circulating tumor cell is present in the at least one biological sample from a patient diagnosed with a type of cancer if at least one mRNA species is present in a substantially greater amount in the at least one biological sample from the patient diagnosed with a type of cancer when compared to the at least one biological sample from the healthy patient.

2. The method of claim 1, wherein the biological sample is a blood sample.

3. The method of claim 1, wherein the biological sample is a bone marrow sample or a lymph node sample.

4. The method of claim 1, wherein the sequence of the mRNA species detected by the method comprises at least a portion of at least one of SEQ ID NOS:1-15.

5. A method of identifying the presence of at least one differentially expressed transcript associated with the presence of at least one circulating tumor cell in a biological sample of a cancer patient, the method comprising the steps of:
providing at least one biological sample from a patient diagnosed with a type of cancer;
providing at least one biological sample from a healthy patient;
lysing the cells present in each of the biological samples without performing any cell separation or cell enrichment steps prior to lysing the cells;
isolating total RNA or poly(A)+ RNA from each of the biological samples;
performing suppression subtraction hybridization to construct a subtracted cDNA library; and
determining that at least one circulating tumor cell is present in the at least one biological sample from a patient diagnosed with a type of cancer if at least one mRNA species is present in a substantially greater amount in the at least one biological sample from the healthy patient when compared to the at least one biological sample from the patient diagnosed with a type of cancer.

6. The method of claim 5, wherein the biological sample is a blood sample.

7. The method of claim 5, wherein the biological sample is a bone marrow sample or a lymph node sample.

8. The method of claim 5, wherein the sequence of the mRNA species detected by the method comprises at least a portion of at least one of SEQ ID NOS:16-23.

9. A method of providing a gene expression profile for a metastatic cancer phenotype, the method comprising the steps of:
providing two or more biological samples from patients diagnosed with a type of cancer and combining such biological samples;
providing two or more biological samples from healthy patients and combining such biological samples;
lysing the cells present in the combined biological samples from patients diagnosed with a type of cancer without performing any cell separation or cell enrichment steps prior to lysing the cells;
lysing the cells present in the combined biological samples from healthy patients without performing any cell separation or cell enrichment steps prior to lysing the cells;
isolating total RNA or poly(A)+ RNA from the combined biological samples from patients diagnosed with a type of cancer;
isolating total RNA or poly(A)+ RNA from the combined biological samples from healthy patients;
performing suppression subtraction hybridization to construct two subtracted cDNA libraries, wherein the first subtracted cDNA library comprises mRNA species present in a substantially greater amount in the combined biological samples from patients diagnosed with a type of cancer when compared to the combined biological samples from healthy patients, and wherein the second subtracted cDNA library comprises mRNA species present in a substantially greater amount in the combined biological samples from healthy patients when compared to the combined biological samples from patients diagnosed with a type of cancer; and
constructing a gene expression profile based on the differentially expressed mRNA species.

10. The method of claim 9, wherein the biological sample is a blood sample.

11. The method of claim 9, wherein the biological sample is a bone marrow sample or a lymph node sample.

12. The method of claim 9, wherein the gene expression profile comprises at least a portion of at least one of SEQ ID NOS:1-23.

13. A gene expression profile for a metastatic cancer phenotype, the gene expression profile constructed by a method comprising the steps of:
providing two or more biological samples from patients diagnosed with a type of cancer and combining such biological samples;
providing two or more biological samples from healthy patients and combining such biological samples;
lysing the cells present in the combined biological samples from patients diagnosed with a type of cancer without performing any cell separation or cell enrichment steps prior to lysing the cells;
lysing the cells present in the combined biological samples from healthy patients without performing any cell separation or cell enrichment steps prior to lysing the cells;
isolating total RNA or poly(A)+ RNA from the combined biological samples from patients diagnosed with a type of cancer;
isolating total RNA or poly(A)+ RNA from the combined biological samples from healthy patients;
performing suppression subtraction hybridization to construct two subtracted cDNA libraries, wherein the first subtracted cDNA library comprises mRNA species present in a substantially greater amount in the combined biological samples from patients diagnosed with a type of cancer when compared to the combined biological samples from healthy patients, and wherein the second subtracted cDNA library comprises mRNA species present in a substantially greater amount in the combined biological samples from healthy patients when compared to the combined biological samples from patients diagnosed with a type of cancer; and
constructing a gene expression profile based on the differentially expressed mRNA species.

14. The gene expression profile of claim 13, further defined as comprising:
detection of at least a portion of at least one of SEQ ID NOS:1-15 in a substantially greater amount in the combined biological sample from patients diagnosed with a type of cancer when compared to the combined biological samples from healthy patients; and
detection of at least a portion of at least one of SEQ ID NOS:16-23 in a substantially greater amount in the combined biological samples from healthy patients when compared to the combined biological samples from patients diagnosed with a type of cancer.

15. A method of treating a patient, comprising the steps of:
constructing a gene expression profile, comprising the steps of:
providing at least one biological sample from the patient;
providing at least one biological sample from a healthy individual;
lysing the cells present in each of the biological samples without performing any cell separation or cell enrichment steps prior to lysing the cells;
isolating total RNA or poly(A)+ RNA from each of the biological samples;
performing suppression subtraction hybridization to construct at least one subtracted cDNA library; and
determining that at least one circulating tumor cell is present in the at least one biological sample from the patient if at least one differentially expressed mRNA species selected from the following is present in the at least one subtracted cDNA library:
(A) at least one mRNA species present in a substantially greater amount in the at least one biological sample from the patient when compared to the at least one biological sample from the healthy individual; and
(B) at least one mRNA species present in a substantially greater amount in the at least one biological sample from the healthy individual when compared to the at least one biological sample from the patient; and
treating the patient with an effective amount of an anticancer agent if at least one differentially expressed mRNA species is detected in the subtracted cDNA library.

16. The method of claim 15, wherein the biological sample is a blood sample.

17. The method of claim 15, wherein the biological sample is a bone marrow sample or a lymph node sample.

18. The method of claim 15, wherein the at least one differentially expressed mRNA species detected comprises at least a portion of at least one of SEQ ID NOS:1-23.

19. A method of identifying circulating tumor cells present in a biological sample from a patient, comprising the steps of:
screening the biological sample for presence of a mRNA species comprising at least a portion of at least one of SEQ ID NOS:1-23.

20. A method of identifying circulating tumor cells present in a biological sample from a patient, comprising the steps of:
screening the biological sample for presence of a mRNA species comprising at least a portion of at least one of SEQ ID NOS:1-15; and
screening the biological sample for absence of a mRNA species comprising at least a portion of at least one of SEQ ID NOS:16-23.

21. A kit, comprising:
an agent for detection of a mRNA species in a biological sample, wherein the mRNA species comprises at least a portion of at least one of SEQ ID NOS:1-15; and
a control sample substantially free of said mRNA species.

22. The kit of claim 21, wherein the agent for detection of a mRNA species detects an expression product of the mRNA species.

23. The kit of claim 21, further comprising a second agent for detection of a second mRNA species in a biological sample, wherein the second mRNA species comprises at least a portion of at least one of SEQ ID NOS:16-23, and wherein the control sample is substantially free of said second mRNA species.

24. The kit of claim 23, wherein the second agent detects an expression product of the second mRNA species.

25. A kit comprising:
an agent for detection of a first mRNA species comprising at least a portion of at least one of SEQ ID NOS:1-15;
an agent for detection of a second mRNA species comprising at least a portion of at least one of SEQ ID NOS:16-23; and
a control sample in which the second mRNA species can be detected, wherein the control sample is substantially free of the first mRNA species.

26. The kit of claim 25, wherein the agent for detection of a first mRNA species detects an expression product of the first mRNA species.

27. The kit of claim 25, wherein the agent for detection of a second mRNA species detects an expression product of the second mRNA species.


SEQUENCE LISTING (35 sequences)

SEQ ID NO: 1 - DNA, 200 bp, Homo sapiens
tctcacgcctataatccccacactttgagaggccaaggtgggcggatcacttgaggtcaggagtttgagaacagcctggccaacatggtgaaaccccatctccactaaacatacaaaaatgagctgggcatggtggcacactcctgtaatcccagctactcgggaggctgaggcaagagaatggcttgaacctgggaggt

SEQ ID NO: 2 - DNA, 191 bp, Homo sapiens
aggtcagcagagatgtctgtatgcttgaaatctatattgttttgctcaggttaatttaatttaatggcaataaatgatttaccaagcacattaaccattaacaccagcaaagtagtcagaagtttaagacatctatctcaagttaatgcaggaaattagaacccccatctttaattctttctcggggacta

SEQ ID NO: 3 - DNA, 232 bp, Homo sapiens
atccaccccaacaatcaagatatgaaagatttccatcacccaagtaattagtttcctcatgcctgtttgtagtcagtctcccccttccccagccagtaagctgcttttgtcattatggttttgccttttctagaatttgataaagatgtgatcatatagtgagtaatcttttgtgctaaacttctttcacttagcatgtttttgagattcagtcccgctggtgtgtgttatt

SEQ ID NO: 4 - DNA, 419 bp, Homo sapiens
accaaacagggcttacaattgtgaattctttctctgtctcttgagatgtatgtgtatctcctgcaagtcaggagtgtctctctcaagaatctgaaagccattcctttgaaatgtaattatcaggaaggatagggccctggctcctaactggtgttaattgccagctagcacgtgcagctggcctaattatggtgatgcccaccaaccgtttgtaaggcttcaccagagtcccacaggctcctcctccctcctcccgcatcttcccctttaaaacgcccagtcacctcagcacaaatcagaattgagctctgctccttctcttactctcagtagttactgaataaaatctgttttcaccacgttaacggacggttgtttatctttgacaataaatactaagatctatcttcccaacctca

SEQ ID NO: 5 - DNA, 432 bp, Homo sapiens
gtaccttcacctaccccccacccatccagaggaataagtcacaccgtggaattcttcgcataaatgacatctgccgtcttaggggtggtctgtaacccttaggagaattttgtgcaaatattccgttgcttctgcattccaggataatgcgttgtgttacggttgttttaggtaaacacactctggcaggaaagccaatatggcaggtgccacataatggggcaaaggtgtgaactcaatgttgaattctttcaacttgctgaggacttggactcaaagggcagatttgaatgggtctagtcccaacaggtgtttctggtttttttgttttgaaagggaaatcataagggaacaattaggcattgtcaggaacatagtcaggaaaaggcaagagtttttaaataattatatgatccacctgaaccatgtacc

SEQ ID NO: 6 - DNA, 756 bp, Homo sapiens
gtacaaaaatatttatcttttaaaacatgcaaaaatttcttgacaaggcacttttaggtataaaatgaagatgagtccttggttctacattcacactgaagtaatagtgaaacatcatcacagctgcactctcaaagccctcagaggtccagcagtctctaaaaactcgtcaacaagactaaaaacattcacggctttacaatgtgggttacagagctttacaaccatgaccaggaaaaactgctcgtaacaacagctgtccttcccagttccacatgtgttgtctcactgcaggaaattaagataagctgccaaattgtctttgtgtgaccctataagatcccatcatccatgtaacttcttgtctccgatttcagtgaacatgtcaggagagacgtgatcgggactctcccaggactacattcaggtgagaaattttagaagaggaaacacaaccaaggttttcatcccctatccattgagagggaagatccagaaaatgggcagttccaaaaaaggaacacttttttgattgataagcatatatggcccatttaatgtccatgactacaactaatgcctcttttcaaaaagaaaatggtttgaaaagatcaaaaccacagagcaatctcctaacagttccaaatttacatcttctaagaatgtataattttgccaccattaaaatgaaatcttaaaggtaaattaaattctcccattgactctgacattgttttaatttctggagaactgtac

SEQ ID NO: 7 - DNA, 753 bp, Homo sapiens
  misc_feature (654)..(654): n is a, c, g, or t
tcgagcggccggcccgggcaggtacttttctgaagtagaggtctgtctttgacttggcaagtccccacaaggtataagaaggcaagcattaaatgcaagtttgaggcgaaattgacttggttatgttaataactagatggtcagcaatagagccaggaaagaagaaagagtaatagaatagatgaaagagttaaatttttcttagctttagtttggtagggttttcccctgggactatggcccacaactctggagcgggtggtgctttcttgactcgggtgtgatgagtccatcccttttttgctgtctggactgcggttttagtggttagaagccgcaggtaaggtccttcccaggccagctcaagtttttcctccttgatgaggacgtgatccccaggctgttattaatggactgagagctccagggttagcgcctgtgctaagagacctttggttttaagaaaaagagaaaataaggcagtctgtagagccatctcataaagggaaaggcctgtatctttttgaggagcagtttggattttcatcagggcaattggaagacatttggtccatggcaactgagtctttataactaatttggttagatggttctttaagctctgatttgtctttttaaccttccttaatgacngtgggtgccacggagtatgatatttttcattttaatgttagggcttgggatagctttttaatgatatgagctgtgaaatgggttccattgtctgagtca

SEQ ID NO: 8 - DNA, 488 bp, Homo sapiens
gtactgttttgtaaaaaaagataaaaagttctgtaatgtgccacattttgagcagtttcttcagcctcaagaatctctggcttgttcaaaacatatctgggtgacactgggaaagttaggacgaggctggaaagaaccaaaggagaggaataattttacaataataataaaggggaaggaaaaaagagttgatgaaatttcacctaaaatgggattaacttatcttgttgaatcaagttatctatttctggtcaatatatttataaaaaacaatagtttaagaagtttcttagaaaaaaaatcttacacaaaagctcctttcattcgtgaaaatacttgtaaatatttttgctttaggagaaaatatgtgctttcagggatattacgctggtctcttaaaagttccttaaaattcttagcttggagtcactgttacttgtaactggaggtattagtgagcctttgggttggacttatgaactatgtac

SEQ ID NO: 9 - DNA, 584 bp, Homo sapiens
gtacaactataacacaaaaaaccctacaagacaggaagggccagggggtcttgagacattttacttggcctcataagaccctcagcaagaagatcacattcctgacccacagcccacatgagccaacaggtgctcagatgtatttccaaggttgcccaagtcacccaaatgaatgacagcattcatttctcaaaaagcccaaggttaggccacagctattcaaccccagtgtaaaacaaaataattggtcaaagtaaaaaccacaactgagacttcagtaaagttaacgggcctcctgtcatttttatgaaggtattgggaatactttaaatgaatgtcaatctgttggatttctgcaatgatgtaatatatatatagaagagtaatttaagctaaaaagcagaaaggagtctctcaaaaaaggaataatatgggagagaagaaatgtgctgacagatgaatgaaactggagaagacagatctctaatggatgttctgttatgtttctgcatccccactgccccaaaagtccatagtaaaagaaacatttccaatttttaaacaaagcagactatggattaaca

SEQ ID NO: 10 - DNA, 165 bp, Homo sapiens
gcgaagttctgcagcactaatatagccattgccatccttatcaaacacacggaatgcttctctaatttcttcttcactgtctgtgtctttcatttttcttgccatcattgtcagaaattcagggaagtcaattgtgccattaccatcagcatctacttcattaat

SEQ ID NO: 11 - DNA, 172 bp, Homo sapiens
taaagcaatatattcaaccttctctagagttggaggttcttggacaaataaactttaaaagggaaaaaaaagcaatatagtcacccttcaaaacagtcaacataataatcatacaattctttggctgagcatagcggttcatgcctgtaatcccagtactttgggaggcctt

SEQ ID NO: 12 - DNA, 191 bp, Homo sapiens
tcaccattttatatagtcttcagttttctaaatatcttgggtttagcagaatcatctctaaaatttagttctactaaatttcctaatgcttcttctcacctcctgtccattactttattttaaacaaatgagtatctaagcgtatacaacaccaccctatttcagtttgatcaaaaggaagtgtcataaaa

SEQ ID NO: 13 - DNA, 431 bp, Homo sapiens
aattcgagacagagatggagacatccagccccacctctctggaacaggaaagatgatcggggagggaacacaggtcagtgtggggacaggggtcacggtggacacgggggtggctgtctctccacctcctcacattatgctaacaggaacgcagacacattcagatgcctttgcagaaagagacgccagaggctcttgaagtcacaaaggagaggtgtgaagaaatcctgcatctcagtcccacacaggcagctgtctcaggctttacaagtgatgagagactcatcagagccctgggcactgttgctgcacgcagcctgagagcagctccctccttttccacctgagctcttcctcctacacatcatagcggtgaccacagctccaaggacagctaggacaaccaggacagccaggccagcaacgatgcc

SEQ ID NO: 14 - DNA, 297 bp, Homo sapiens
agggaagtgcctgtgtccacaatggcctcacagccctccttgcacagggtcagcccgctggccacctccacctggtccaggtggacctgccagtaggccttgcgggtgacattcaggtaggacagagaacccttgtaatacttggagtccgtgccacccagcatcagctcacccccaggctgcgcatctgggtccctgctcaggtagaaggagaagatgttctggtccaccagcttctgctgcatcaggttgtcgaagacagggcagcacgttgttgacggagatgcgggggtaggc

SEQ ID NO: 15 - DNA, 420 bp, Homo sapiens
  misc_feature (375)..(375): n is a, c, g, or t
atgcaactgataggataagttttccatcaatacttcgacccatagatagattgaccatttgcaatacttaatagcaagcaattgtagcaccactaatttttcatttatataatattaaggaagacacccatattttaaattcaattcctttcttaagagactggaattcttccattcttgatgcttatgccttgaaaaattgtcactcttcaaaacagaaaaggacttcagaaacataatgactagtgaatacaagttaaaaattaaaagtaacacaagtgatgttattttcccttaaagcaaaatactatatttaaaatgcagtgttttatccgttttcctggatcacctgataataccattgtgtatataagngatacttagaaaatgggagtttgggccaggcgcgatggctcatgt

SEQ ID NO: 16 - DNA, 194 bp, Homo sapiens
tgacaagtcagggtgaaagctggttgcagtggcacacggccagaaattaatggaaattctctatgatgatacccatagcggcaaagaacaatgcatatgccacacaaaatcctatagatcatctcttctaacgcagtttggttcaacaagtaactgcgtgcctattactcaacagaagtgtgttgagtgcccct

SEQ ID NO: 17 - DNA, 429 bp, Homo sapiens
gtacaacttatagaaaaggtaaaggaaaccccaacatgcatgcactgccttggtgaccagggaagtcaccccacggctatgggaaattagccccgagggttagctttcattatcactgtctcccagggtgtgcttgtcaaagagatattccgccaagccagattcgggcgctcccatcttgcgcaagttggtcacgtggtcacccaattctttgatggctttcacctgctcattcaggtaatgtgtctcaatgaagtcacacaaatgggggtcatttttgtcagtggccagtttgtgcagttccagtagtgactgattcacatttttttccaaatgtaatgcacactccattgcattcagcccgctctcccagtcatcacagtctggtttcttgatatcctgaaggaagattcggccacctcgttggtt

SEQ ID NO: 18 - DNA, 181 bp, Homo sapiens
acaggatagtgcctatagttaacagtattgtattgtatacttaaatatttgctaagatggtagatcttatgtaaaattttcttatcacacacaaaatagtagaaataataaagagagcagaaagaaacttctggaggtgataggtttatggcatagattgtggtgatggtttcatgagtgt

SEQ ID NO: 19 - DNA, 213 bp, Homo sapiens
tcagatccatccatgcattgcttcatctcaaatgtgaaagacattatatagtattgagacaaaataataatgttgtgacaaaccaacgaagtaaagacatttagaattaagtttgacaattcttaactgagtccactatactctggtattataaaatatgcatgttatctatggacacaaaagagcaaggaaagggcagtgtgtcagtattga

SEQ ID NO: 20 - DNA, 138 bp, Homo sapiens
agggaatggccaagtaagtatcaatcagttcccaaattaatcttggaggttattttgagaggtttagtcctaccctcagaatttcacaaataattcctttgtgcaatccatgtaagaaatgttctctgaaatcacact

SEQ ID NO: 21 - DNA, 195 bp, Homo sapiens
agcatgagttaccatgcctgtgatggttttaccaattggttctttttacgccttttcttataaattttagggttgatttgtcaatttctaagagaaaagaatctgagatcttgattgggattatgttgaatgtatagatcaattgaaaaggaattagcactttaagaatacagagccttcaaccaatgcatgtgt

SEQ ID NO: 22 - DNA, 430 bp, Homo sapiens
  misc_feature (35)..(35): n is a, c, g, or t
  misc_feature (38)..(38): n is a, c, g, or t
  misc_feature (74)..(74): n is a, c, g, or t
  misc_feature (90)..(90): n is a, c, g, or t
  misc_feature (107)..(107): n is a, c, g, or t
  misc_feature (158)..(158): n is a, c, g, or t
  misc_feature (179)..(179): n is a, c, g, or t
  misc_feature (194)..(194): n is a, c, g, or t
  misc_feature (240)..(240): n is a, c, g, or t
  misc_feature (260)..(260): n is a, c, g, or t
  misc_feature (270)..(270): n is a, c, g, or t
  misc_feature (299)..(299): n is a, c, g, or t
  misc_feature (353)..(353): n is a, c, g, or t
  misc_feature (358)..(358): n is a, c, g, or t
  misc_feature (362)..(363): n is a, c, g, or t
  misc_feature (368)..(368): n is a, c, g, or t
  misc_feature (386)..(387): n is a, c, g, or t
  misc_feature (393)..(393): n is a, c, g, or t
  misc_feature (397)..(397): n is a, c, g, or t
  misc_feature (415)..(415): n is a, c, g, or t
  misc_feature (422)..(422): n is a, c, g, or t
  misc_feature (427)..(427): n is a, c, g, or t
acatgaagactttgatcccttgacaaatgtcattngtnggtgagcaattattaataggattgtaagtcttaatntgcaaggatcaccatnaataaaagtagattatngtaaagtgaattagcaataattttacttatgaataaaaacttatactattncaggtcattttcttattgttncttgaaaaaagaagncctttataagtccagttaaatattacctaatttttgcatcaaattncatggtaaacaaggcttatnttcttacttnactgatgtctaaaactaaatttcactggngatatatcatttctttaagactaggtaactgtaacctaagctgagacccaaaanttgtnattnnctctnttttacagcacaatattnnccatcntggnatattacatcttgctaangaagctntgttnctt

SEQ ID NO: 23 - DNA, 280 bp, Homo sapiens
agcacaatgggtcaacaagaagtttacatttaagaattattaaataatactttaaatggaagtaaaagcagtcaatggtaacagaccctgaatttttctttcaaatgctcacacttactttgtctagtgtcatgtctggtagttcatctgccagttcacttggggaactatttgcactggaatttgcaactgctggaattgtaggttcatagccatagaatgccagtaaatcttctagaggcatggttccttcctgttcaagccagggcataaacacgga

SEQ ID NO: 24 - DNA, 21 bp, Homo sapiens
aggaataagtcacaccgtgga

SEQ ID NO: 25 - DNA, 21 bp, Homo sapiens
acctgttgggactagacgcat

SEQ ID NO: 26 - DNA, 21 bp, Homo sapiens
tggtctgtaacccttaggaga

SEQ ID NO: 27 - DNA, 20 bp, Homo sapiens
tctgccctttgagtccaagt

SEQ ID NO: 28 - DNA, 20 bp, Homo sapiens
aggtcagcagagatgtctgt

SEQ ID NO: 29 - DNA, 20 bp, Homo sapiens
tagtccccgagaaagaatta

SEQ ID NO: 30 - DNA, 21 bp, Homo sapiens
tgagcagtttcttcagcctca

SEQ ID NO: 31 - DNA, 21 bp, Homo sapiens
agagaccagcgtaatatccct

SEQ ID NO: 32 - DNA, 21 bp, Homo sapiens
tatctgggtgacactgggaaa

SEQ ID NO: 33 - DNA, 21 bp, Homo sapiens
agagaccagcgtaatatccct

SEQ ID NO: 34 - DNA, 24 bp, Homo sapiens
aggtaaaggaaaccccaacatgca

SEQ ID NO: 35 - DNA, 23 bp, Homo sapiens
aaccaacgaggtggccgaatctt